SEQUENCE LISTING (6 sequences)

SEQ ID NO: 1 - DNA, 30 bp, Artificial Sequence
  Synthetic Marker
catgtaagaggttggtctcctttggcggga

SEQ ID NO: 2 - DNA, 48 bp, Artificial Sequence
  Synthetic Marker
gatcgtcggactgtagaactctgaacctgtcggtggtcgccgtatcat

SEQ ID NO: 3 - DNA, 43 bp, Artificial Sequence
  Synthetic Marker
caagcagaagacggcatacgattttttttttttttttttttvn

SEQ ID NO: 4 - DNA, 20 bp, Artificial Sequence
  Synthetic Primer
caagcagaagacggcatacg

SEQ ID NO: 5 - DNA, 46 bp, Artificial Sequence
  Synthetic Primer
aatgatacggcgaccaccgacaggttcagagttctacagtccgacg

SEQ ID NO: 6 - DNA, 151 bp, C. elegans
gactgcagtcgtacgactaggccctagctgcgtacgatgcgctcgatcaacgtttgcagcttacgatgcaccgtgacaccgctgctacgtacatgatcgtgactgtatgtcagtacaccatacccgtgctcagtcacacgtcgtcgtcgta

(19) United States
(12) Patent Application Publication
(10) Pub. No.: US 2013/0149699 A1
(43) Pub. Date: Jun. 13, 2013

(54) Translation Kinetic Mapping, Modification and Harmonization

(71) Applicants: The Board of Trustees of the Leland Stanford Junio, Palo Alto, CA (US); The University of Texas Medical Branch at Galvest, Galveston, TX (US)

(72) Inventors: Jose M. Barral, Galveston, TX (US); Andrew Fire, Stanford, CA (US); Paige S. Spencer, Galveston, TX (US); Michael Stadler, San Francisco, CA (US)

(73) Assignees: The University of Texas Medical Branch at Galveston, Galveston, TX (US); The Board of Trustees of the Leland Stanford Junior University, Palo Alto, CA (US)

(21) Appl. No.: 13/664,252

(22) Filed: Oct. 30, 2012

Related U.S. Application Data
(60) Provisional application No. 61/553,861, filed on Oct. 31, 2011.

(51) Int. Cl. C07H 21/02

(52) U.S. Cl. CPC C07H 21/02
USPC 435/6.1; 536/25.3; 435/69.1; 536/23.1; 435/68.1

(57) ABSTRACT

The profile of translation elongation rate along an mRNA is modulated in a directed manner by locally altering codon usage, in particular utilizing differences in ribosomal dwell times among pairs of synonymous codons translated by a single tRNA through wobble base pairing. Unlike codon optimization based on organism-specific codon frequencies or tRNA pools, the methods of the invention need not change the tRNA that translates the codon, rather modulating the interaction between a given tRNA and the mRNA coding sequence.

### GOVERNMENT RIGHTS

This invention was made with Government support under contracts GM037706 and A1065359 awarded by the National Institutes of Health. The Government has certain rights in this invention.

### BACKGROUND OF THE INVENTION

Though most amino acids can be encoded by multiple RNA sequences (i.e., “codons”), organisms sample from this sequence space in a biased manner, with individual codons showing species-specific over- or under-representation. One biological consequence of codon choice is to influence local translation elongation rates on messenger RNAs. The rate of translation elongation can influence both the amount and quality (e.g. folding) of protein produced, indicating that the specific codon sequence, beyond merely dictating the amino acid sequence, can be an important determinant of the final biological output of a gene.

There are codon-optimization techniques available for improving the translational kinetics of translationally inefficient protein coding regions. Many of these techniques rely on identifying the codon usage for an organism where the intended translation will occur (the “host” organism), and altering codons from the intended coding region (the “donor” coding region) to encode the same protein but contain a substantially increased proportion of codons which are the most common (or among the most common) for each amino acid in the host's transcriptome. Deficits in this approach include (i) that relative codon frequencies are not necessarily a precise predictor of translation rate, and (ii) that maximum rates of translational elongation may in some cases not be optimal for folding of active protein.

Complex associations between folding rate landscapes and active protein yields have been both hypothesized and observed. Indeed, optimal synthesis of a functional protein that folds co-translationally could require a folding landscape in which individual segments of the polypeptide are allowed extra time (or less time) during the synthesis of the polypeptide by ribosomes. This would generate a situation in which adjustments to the translation rate for specific regions of the polypeptide (both increased and decreased elongation rates) may allow for the most efficient synthesis of functional product. Inadequate coupling between the rates of protein synthesis and folding, often observed during recombinant protein production in heterologous hosts, can result in protein misfolding and aggregation. For example, a subtle increase in the concentration of a partially folded intermediate during translation of its polypeptide sequence may exceed the critical concentration of the intermediate and lead to its nucleation-dependent aggregation, thus forming intracellular aggregates or pathogenic inclusion bodies.

Achieving such a “tailored” kinetic landscape of translation would require a detailed view of the relationship between individual codon choices and organism-specific and cell-specific translation rates. Experiments in microorganisms have demonstrated that a codon's translation rate correlates positively with both frequency of usage and acceptor tRNA concentration, consistent with models in which tRNA selection on the ribosome is among rate-limiting factors in elongation. However, rare codons can, in some cases, be decoded rapidly, and conversely, frequent codons slowly. Codons translated by identical tRNAs using different base-pairing and base stacking geometries can also be decoded at different rates. While these results were derived using limited sets of codons in specific sequence contexts, a systematic assessment of codon translation elongation rates examining many transcripts and diverse sequence contexts can now be achieved (as described in this invention).

Among the effects of codon choice on elongation kinetics during translation, we identified third-base wobble codon pairing as an important example. The G·U wobble base pair is a fundamental unit of RNA secondary structure that is present in nearly every class of RNA from organisms of all three phylogenetic domains. It has comparable thermodynamic stability to Watson-Crick base pairs and is nearly isomorphic to them. Therefore, it often substitutes for G·C or A·U base pairs. The G·U wobble base pair also has unique chemical, structural, dynamic and ligand-binding properties, which can only be partially mimicked by Watson-Crick base pairs or other mispairs. Protein, RNA enzymes and RNA-binding proteins bind to unique G·U sites by recognizing chemical features common to all G·U pairs and that differ from Watson-Crick and other mismatched pairs. Conformational features of G·U-containing RNA double helices unique to the specific sequence context of each G·U pair provide diversity and allow a variety of unique interaction sites to be built from this recognition tag. The presence and distribution of codons with G·U base pairing thus provides an example of sequence features that can be used in a directed manner to adjust kinetics of translation in a desired manner.

It is an object of the present invention to provide new methods for codon optimization that expand the capability or circumvent the drawbacks of the prior art.

### PUBLICATIONS

Palidwor et al. 2010. A general model of codon bias due to GC mutational bias. PLoS ONE 5: e13431. Nussinov 1981. Eukaryotic dinucleotide preference rules and their implications for degenerate codon usage. Journal of molecular biology 149: 125-31. Crick 1966. Codon—anticodon pairing: the wobble hypothesis. Journal of molecular biology. 19: 548-555. Crombie et al. 1992. Protein folding within the cell is influenced by controlled rates of polypeptide elongation. Journal of molecular biology 228: 7-12. Ingolia et al. 2009. Genome-wide analysis in vivo of translation with nucleotide resolution using ribosome profiling. Science 324: 218-23. Tuller et al. 2010. An evolutionarily conserved mechanism for controlling the efficiency of protein translation. Cell 141: 344-54. Sorensen & Pedersen (1991) J Mol Biol 222, 265-80. Curran & Yarus (1989) J Mol Biol 209, 65-77. Gupta et al. (2000) Biochem Biophys Res Commun 269, 692-6. Thanaraj & Argos (1996) Protein Sci 5, 1973-83. Thanaraj & Argos (1996) Protein Sci 5, 1594-612. Angov E, Hillier C J, Kincaid R L, Lyon J A, 2008 Heterologous Protein Expression Is Enhanced by Harmonizing the Codon Usage Frequencies of the Target Gene with those of the Expression Host. PLoS ONE 3(5): e2189. doi:10.1371/journal.pone.0002189

### SUMMARY OF THE INVENTION

Compositions and method are provided for modulating the translation kinetics of an mRNA, or a region or domain of an mRNA, by appropriate selection of codons, particularly with respect to “wobble” codon pairs. In some embodiments of the invention, the rate of translation elongation is altered, i.e. decreased or increased, by appropriate selection of specific codons for each amino acid of the encoded polypeptide chain. In some cases, the increase or decrease in elongation rate may be through the choice of one member of a wobble codon pair, which does not change the tRNA that translates the codon, but rather modulates the interaction between a given tRNA and the mRNA coding sequence. A coding sequence may be modified to increase or decrease translation speed at a position (or positions) of interest.

In some embodiments of the invention, methods are provided for determining the “kinetic map” of translation for an mRNA in an organism of interest. The kinetic map accounts for the host-cell-specific speed of translation elongation for individual codons. Typically there is a variation in speed over the length of the mRNA, where the variation will depend on factors including the tRNA pool size and the use of wobble codons by the host cell.

In other embodiments a coding sequence of interest is modified to “harmonize” the translation kinetic map between the source organism and an intended organism for expression. Such methods include the step of determining a kinetic map for the mRNA in the source organism through experimental examination of polypeptide synthesis or inference based on codon choices and tRNA repertoire; determining the appropriate codon selections in the expression host cell system that will match the translation speed of the source organism; and synthesizing de novo or modifying the existing coding sequence to provide for a harmonized speed. Such a sequence may be referred to as a “harmonized” sequence, or as a sequence harmonized for an organism of interest.

The invention also includes methods of producing a protein encoded by a sequence modified by the methods of the invention, including translation harmonized sequences. In some embodiments, the modified sequence provides for increased synthesis of active, i.e. properly folded, protein, and may increase the yield of active protein by greater than 2-fold relative to the native sequence, particularly where there is a significant evolutionary distance between the source organism and the expression host organism, e.g. between eukaryotes and prokaryotes, or between distantly related prokaryotic or distantly related eukaryotic species.

Nucleotide sequences are provided in which the codon usage of the nucleotide sequence has been selected with respect to “wobble” codon pairs or other cases where there is substantial difference in ribosomal dwell times between synonymous codons Such sequences may be harmonized sequences, as described above, or may be altered to decrease or increase translation elongation rate at positions of interest without harmonizing. In some embodiments, one or more codons have substituted relative to a native sequence, which substitutions may increase translation elongation rate, decrease the rate, and/or be designed to optimize desired translation kinetics by selection of the appropriate codon, particularly when a codon is subject to increased or decreased ribosomal dwell time, such as is observed for members of a wobble codon pair.

In some embodiments, methods are provided for increasing the expression of a protein, by expressing a polypeptide-encoding sequence that has been selected in codon usage to decrease the number of codons having wobble pairing during translation. In particular, the method comprises the step of expressing a nucleotide sequence selected for wobble codon usage, which encodes at least one polypeptide. Expression systems of interest include cells and cell-free translation systems. The selected nucleotide sequence can be derived from a different starting nucleotide sequence such that the codon usage of the modified nucleotide sequence is adjusted to reduce the number of codons that rely on wobble pairing, or can be a synthetic sequence selected to reduce the number of wobble codons. Generally the amino acid sequence is not altered by the selection for or against wobble codons.

In other embodiments, methods are provided for decreasing the speed of translation elongation of an mRNA or a region of an mRNA, by expressing a polypeptide-encoding sequence that has been selected in codon usage to increase the number of codons having wobble pairing during translation. Such methods find use, for example, in the translation of proteins that are improperly folded during expression of the native sequence, and which may increase in stability or correct folding by slower translation kinetics. Sequences may be further optimized by modifying the wobble codon usage to increase translation rate in certain domains, for example fast-folding domains, and decreasing the translation rate in other domains.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1. Positioning ribosome sites and analysis overview. (A) A single tRNA family with G in the wobble position can use two types of base-pairing to decode synonymous codons. (B-C) Cumulative coverage of RPF start nts mapping near the initiation codon. Data shown is the aggregate normalized (per transcript) coverage across all mRNAs in (B) C. elegans and (C) HeLa datasets. Individual colored lines represent independent biological and technical replicates. Arrows indicate the peak inferred to represent the position of RPFs with the AUG in the P-site. (D) Location of tRNA binding-sites and structure of RPFs inferred from data in A-B. (E) Overview of analytical approach for determining ribosome occupancy for codon-ribosomal site combinations.

FIG. 2. Ribosome occupancy for C/U-ending codon pairs in C. elegans. (A) Wobble-position base-pairing geometries used by C/U-ending codons are shown in the top-left panel. Remaining panels show the difference in ribosome occupancy for synonymous C/U-ending codon pairs (NNU/NNC). Codons that are read by G:C/G:U base-pairing at the wobble position are shaded black, pairs read by I:C/I:U are shaded gray. Error bars represent the standard deviation. (B) Identical figure showing control occupancy data for alkali-sheared mRNA.

FIG. 3. Ribosome occupancy for C/U-ending codon pairs in HeLa cells. Panels are as in FIG. 2. White bar indicates that for this codon pair, the human genome encodes multiple copies of cognate tRNAs with anticodons with A and G in the wobble position; codons may therefore not share a common tRNA pool. (A) data from RPF libraries, (B) data for sheared mRNA.

FIG. 4. Wobble-position ribosome occupancy effects are consistent on different regions of mRNAs in C. elegans. Panels are as in FIG. 2. The transcriptome was divided into three sequence sets: (A) the first 50 codons of each message, (B) the last 50 codons, and (C) all coding sequence excluding the first and last 50 codons. Coding sequences that were not long enough to accommodate the partitioning were excluded. Ribosome occupancy calculations were carried out individually on each sequence set identically to calculations shown in FIG. 2.

FIG. 5. Wobble-position ribosome occupancy effects are consistent on different regions of mRNAs in HeLa cells. Panels are as in FIG. 2, and data is derived as described for C. elegans in FIG. 4.

FIG. 6. Highly-expressed C. elegans genes avoid slowly-translated wobble codons. (A-B) Preference for NNC vs. NNU codons for individual genes plotted against expression levels measured by whole-animal mRNA-seq, averaged across four larval stages. Genes encoding ribosomal proteins are highlighted yellow. Preference is shown separately for codons read by G:C/G:U at the wobble position (A) and those read by I:C/I:U (B). (C) Preference for NNA vs. NNG among synonymous G/A-ending codons plotted against expression level. (D) Table of the most NNC-favoring C. elegans genes from (A) with preference significant at p<0.05 (Bonferroni-corrected). Genes are listed in descending order with the gene showing the greatest preference for NNC codons listed first.

FIG. 7. The distribution of genes encoding tRNAs of different decoding capacities vary among archaea, bacteria and eukarya. Predicted gene content for tRNAs capable of decoding the standard genetic code according to GtRNAdb is plotted for each codon in histogram form (as indicated) by each domain of life in different colors (as indicated). The length of each box represents the extent to which genes for tRNAs capable of decoding the corresponding codon are present in a domain. For example, for Ala, no eukaryotic genera examined contain tRNA genes capable of decoding GCC, whereas ˜60%, ˜25% and ˜15% of them contain tRNA genes to decode GCU, GCA and GCG, respectively. For Met or Trp, 100% of genera examined in each domain are predicted to contain a single species of tRNA genes to decode these codons (and thus the length of these bars corresponds to “100% exclusivity”).

FIG. 8. Relative polypeptide elongation rates can be predicted for any mRNA based on the tRNA gene content of the expression host. Plots depicting predicted translation speed indices (see main text and Materials and methods), calculated for the sequence encoding firefly luciferase, utilizing the tRNA gene content of the organisms indicated obtained from GtRNAdb₁₂. Regions with high i values are predicted to be translated rapidly, whereas regions with lower i values are predicted to be translated more slowly.

FIG. 9. Translation rates can be accelerated by engineering a sequence to contain only codons predicted to be decoded by abundant tRNAs. (a) Autoradiograms of SDS-PAGE gels from pulse-chase experiments of live E. coli cells synthesizing recombinant firefly luciferase from the indicated sequence-engineered constructs (see main text and Materials and methods). (b) Plots depicting the appearance of incorporated [³⁵S]methionine in full length firefly luciferase produced from the indicated constructs (colored dots; values obtained by denistometric analysis of the data in a) and curves for the theoretical appearance of methionine with three calculated average translation rates, as indicated (full, broken and dotted lines). (c) Plot of the predicted polypeptide elongation rates for luciferase synthesized from the constructs indicated, calculated as in FIG. 2 (see main text and Materials and methods). Straight broken lines represent the average predicted translation rates over the entire sequence (avg.), as indicated.

FIG. 10. Sequence-based translation acceleration is not due to changes in initiation rates. (a) Autoradiograms of SDS-PAGE gels from pulse-chase experiments of live E. coli cells synthesizing recombinant firefly luciferase from the indicated sequence-engineered constructs (see main text and Materials and methods). (b) Plots depicting the appearance of incorporated [³⁵S]methionine in full length firefly luciferase produced from the indicated constructs (filled dots; values obtained by denistometric analysis of the data in a) and curves for the theoretical appearance of methionine with three calculated average translation rates, as indicated (full, broken and dotted lines).

FIG. 11. Sequence-based acceleration of translation rates affects the folding of the encoded polypeptide. SDS-PAGE of steady state accumulation of total full-length recombinant protein produced in E. coli from the Luc_{WT}, Luc_{fast} and Luc_{cbf} constructs (top panel). Histogram depicting the specific activities of the luciferase protein produced from the indicated constructs. The value of the protein from the wild-type sequence was set to 100. R.L.U. Error bars represent standard errors of the mean (middle panel). SDS-PAGE documenting total (T), soluble (S) and insoluble (P) recombinant protein produced in E. coli from the indicated sequence-engineered constructs (bottom panel).

FIG. 12. Sequence harmonization based on tRNA gene content between the original and the expression host increases folding efficiency. (a) Plots depicting predicted translation speed indices (see main text and Materials and methods), calculated for firefly luciferase translated from a harmonized redesigned sequence (Luc_{RE}) upon expression in E. coli and for the original (Luc_{WT}) sequence expressed in D. melanogaster (see main text and Materials and methods). (b) Histogram depicting firefly luciferase activity (top panel) and SDS-PAGE documenting total (T), soluble (S) and insoluble (P) recombinant protein (bottom panel) produced in E. coli from the indicated sequence-engineered constructs. R.L.U.: relative light units. Bars represent standard errors of the mean.

### DETAILED DESCRIPTION OF THE EMBODIMENTS

The translation kinetics of an mRNA, or a region or domain of an mRNA, is modified by appropriate selection of codons, particularly with respect to “wobble” codon pairs. In some embodiments of the invention, the rate of translation elongation is altered, i.e. decreased or increased, by appropriate selection of one member of a wobble codon pair, which methods do not change the tRNA that translates the codon, but rather modulate the interaction between a given tRNA and the mRNA coding sequence.

Methods are provided for determining the “kinetic map” of translation for an mRNA in an organism of interest. The kinetic map accounts for the organism-specific speed of translation over the length of an mRNA. Typically there is a variation in speed over the length of the mRNA, where the variation will depend on factors including the tRNA pool size and the use of wobble codons by the organism.

A coding sequence of interest may be modified to “harmonize” the translation kinetic map between the source organism and an intended organism for expression. Such methods include the step of determining a kinetic map for the mRNA in the source organism; determining the appropriate codon selections in the expression host organism that will match the translation speed of the source organism; and synthesizing de novo or modifying the existing coding sequence to provide for a harmonized speed. Such a sequence may be referred to as a “harmonized sequence, or as a sequence harmonized for an organism of interest.

As used herein the term “wobble codon pair” refers to a pair of codons that (a) translate to the same amino acid; (b) have identical first two bases; (c) have C or U at the third position; and (d) are translated by a single cognate tRNA with a G in the anticodon wobble position; or in some instances with an A that is modified to inosine (I) in the anticodon wobble position. It is shown herein that in a pair, the codon that has G·C pairing (or I·C pairing), in the third position is translated more rapidly by the ribosome relative to the codon that has G·U wobble pairing (or I·U wobble pairing). Thus, selection of codons that provide for G·C pairing instead of codons that provide for G·U pairing increases the speed with which ribosomes transit an mRNA, or a region or domain of an mRNA; while selection of codons that provide for G·U pairing instead of codons that provide for G·C pairing decreases the translation speed of an mRNA, or a region or domain of an mRNA. Similar results may be obtained with I·C and I·U pairing.

In organisms other than metazoans, for example prokaryotes, fungi, etc., a wobble codon pair may have the properties of (a) translate to the same amino acid, (b) have identical first two bases, and (c) are translated by a common tRNA with pairing achieved through non-Watson-Crick interactions.

It will be understood by one of skill in the art that a codon pair that meets the requirements for a G·U wobble codon can be readily determined by one of skill in the art upon review of the genetic code, and the tRNAs that are present for a species of interest, using, for example a database as described by Chan and Lowe (2009) N.A.R. 37:D93-D97, herein specifically incorporated by reference.

In human cells, the following are G·U wobble codon pairs:

| G•C | G•U wobble | amino | tRNA |
|---|---|---|---|
| codon | codon | acid | anticodon |
| GGC | GGT | Gly | GCC |
| AGC | AGT | Ser | GCT |
| TTC | TTT | Phe | GAA |
| AAC | AAT | Asn | GTT |
| GAC | GAT | Asp | GTC |
| CAC | CAT | His | GTG |
| TAC | TAT | Tyr | GTA |
| TGC | TGT | Cys | GCA |
| ATC | ATT | Ile | GAT/AAT |

In such a codon pair, the codon that has G·C pairing in the third position has increased speed of translation relative to the codon that has G·U wobble pairing. Thus, selection of a codon that provides for G·C pairing over a codon that provides for G·U pairing increases the translation elongation rate over an mRNA, or a region or domain of an mRNA; while selection of a codon that provides for G·U pairing over a codon that provides for G·C pairing decreases the translation elongation rate over an mRNA, or a region or domain of an mRNA.

For some purposes, wobble codon pairs of interest include an “I·U wobble codon pair”, which refers to a pair of codons that have the properties: (a) translate to the same amino acid; (b) have identical first two bases; (c) have C or U at the third position; and (d) are translated by a single cognate tRNA with an A in the anticodon wobble position, which A is edited to isosine to produce a bifunctional tRNA population recognizing both terminal C and terminal U codons at the wobble position.

In human cells, the following are I·U wobble codon pairs:

| I•C | I•U wobble | amino | tRNA |
|---|---|---|---|
| codon | codon | acid | anticodon |
| GCC | GCT | Ala | AGC |
| CCC | CCT | Pro | AGG |
| GTC | GTT | Val | AAC |
| TCC | TCT | Ser | AGA |
| CGC | CGT | Arg | ACG |
| CTC | CTT | Leu | AAG |
| ACC | ACT | Thr | AGT |

In such a codon pair, the codon that has I·C pairing in the third position is generally translated faster relative to the codon that has I·U wobble pairing. Thus, selection of a codon that provides for I·C pairing rather than a codon that provides for I·U pairing increases the translation elongation rate over an mRNA, or a region or domain of an mRNA; while selection of a codon that provides for I·U pairing over a codon that provides for I·C pairing decreases the translation elongation rate over an mRNA, or a region or domain of an mRNA.

Polynucleotide.

As used herein, the term “polynucleotide” is given its common meaning, that is, a polymer of nucleotides linked by phosphodiester bonds, which can hybridize with naturally occurring nucleic acids in a sequence specific manner analogous to that of two naturally occurring nucleic acids. Polynucleotides include naturally occurring adenine, guanine, cytosine, thymidine and uracil, and may also include 2′-position sugar modifications; propynyl additions, for example at the at the 5 position of pyrimidines; 5-position pyrimidine modifications, 7- or 8-position purine modifications, modifications at exocyclic amines, 5-methyl cytosine; 5 bromo-cytosine; alkynyl uridine and cytosine; inosine, substitution of 4-thiouridine, substitution of 5-bromo or 5-iodo-uracil; etc., and includes unusual base-pairing combinations.

The terms “ribonucleic acid” and “RNA” as used herein mean a polymer composed of ribonucleotides. The terms “deoxyribonucleic acid” and “DNA” as used herein mean a polymer composed of deoxyribonucleotides.

A “native sequence” polynucleotide is one that has the same nucleotide sequence as a polynucleotide derived from nature. Such native sequence polynucleotides can be isolated from cells, or can be produced by recombinant or synthetic means. Thus, a native sequence polynucleotide can have the nucleotide sequence of, e.g. naturally occurring human, murine, or other mammalian species, or from non-mammalian species, e.g. Drosophila, C. elegans, prokaryotic cells, and the like.

The term “native sequence polynucleotide” includes the native polynucleotide with or without the initiating N-terminal methionine (Met), and with or without the native signal sequence, and with our without non-coding sequences, e.g. introns, 5″ untranslated regions, 3′ untranslated regions, and the like.

An “artificial” coding sequence is one that encodes a polypeptide, where the polypeptide may be a known protein or peptide or a variant thereof, but where the nucleotide sequence has been created de novo by selection of appropriate codons. For example, see Dahiyat and Mayo (1997) Science 278:82-87, herein specifically incorporated by reference. Methods for synthesis include oligonucleotide synthesis from digital genetic sequences and subsequent annealing of the resultant fragments, without a requirement for an existing DNA template. Redesigning coding sequences for proteins of interest can offers a means to improve gene expression, and/or stability and correct folding of the desired protein. A software program may be utilized for the codon selection process, for example as described by Puigbo et al. (2007) Nucleic Acids Research 35:W126-W131, herein specifically incorporated by reference, or utilizing commercial programs available for this purpose.

Expression Construct:

In the present methods, polynucleotides that have been altered from the native sequence (by changing one or more triplet codons to synonymous triplets encoding an identical amino acid), or artificially created with a selection of codons, can be used to produce polypeptides by recombinant methods. The polynucleotide coding sequence may be inserted into a replicable vector for expression. Many such vectors are available. The vector components generally include, but are not limited to, one or more of the following: an origin of replication, one or more marker genes, an enhancer element, a promoter, and a transcription termination sequence. Construction of suitable vectors containing one or more of the above-listed components employs standard techniques.

Included as polypeptides of interest are those that have the sequence of a native protein, proteins that have been altered from the native sequence, e.g. as a fusion polypeptide with a heterologous polypeptide, e.g. a signal sequence or other polypeptide having a specific cleavage site at the N-terminus of the mature protein or polypeptide; by targeted or random amino acid substitution, by truncation of the native sequence, and the like.

Nucleic acids are “operably linked” when placed into a functional relationship with another nucleic acid sequence. For example, DNA for a signal sequence is operably linked to DNA for a polypeptide if it is expressed as a preprotein that participates in the secretion of the polypeptide; a promoter or enhancer is operably linked to a coding sequence if it affects the transcription of the sequence; or a ribosome binding site is operably linked to a coding sequence if it is positioned so as to facilitate translation. Generally, “operably linked” means that the DNA sequences being linked are contiguous, and, in the case of a secretory leader, contiguous and in reading phase. However, enhancers do not have to be contiguous. Linking is accomplished by ligation at convenient restriction sites. If such sites do not exist, the synthetic oligonucleotide adapters or linkers are used in accordance with conventional practice.

Expression vectors may contain a selection gene, also termed a selectable marker. This gene encodes a protein necessary for the survival or growth of transformed host cells grown in a selective culture medium. Host cells not transformed with the vector containing the selection gene will not survive in the culture medium. Typical selection genes encode proteins that (a) confer resistance to antibiotics or other toxins, e.g., ampicillin, neomycin, methotrexate, or tetracycline, (b) complement auxotrophic deficiencies, or (c) supply critical nutrients not available from complex media.

Expression vectors will contain a promoter that is recognized by the host organism and is operably linked to the coding sequence. Promoters are untranslated sequences located upstream (5′) to the start codon of a structural gene (generally within about 100 to 1000 bp) that control the transcription and translation of particular nucleic acid sequence to which they are operably linked. Such promoters typically fall into two classes, inducible and constitutive. Inducible promoters are promoters that initiate increased levels of transcription from DNA under their control in response to some change in culture conditions, e.g., the presence or absence of a nutrient or a change in temperature. A large number of promoters recognized by a variety of potential host cells are well known. Transcription by higher eukaryotes is often increased by inserting an enhancer sequence into the vector. Enhancers are cis-acting elements of DNA, usually about from 10 to 300 bp, which act on a promoter to increase its transcription. Enhancers are relatively orientation and position independent, having been found 5′ and 3′ to the transcription unit, within an intron, as well as within the coding sequence itself. Many enhancer sequences are known from mammalian genes. Examples include the SV40 enhancer on the late side of the replication origin, the cytomegalovirus early promoter enhancer, the polyoma enhancer on the late side of the replication origin, and adenovirus enhancers. The enhancer may be spliced into the expression vector at a position 5′ or 3′ to the coding sequence, but is preferably located at a site 5′ from the promoter.

Expression vectors used in eukaryotic host cells (yeast, fungi, insect, plant, animal, human, or nucleated cells from other multicellular organisms) will also contain sequences necessary for the termination of transcription and for stabilizing the mRNA. Such sequences are commonly available from the 5′ and, occasionally 3′, untranslated regions of eukaryotic or viral DNAs or cDNAs. These regions contain nucleotide segments transcribed as polyadenylated fragments in the untranslated portion of the mRNA encoding Wnt polypeptide.

Suitable host cells for cloning or expressing the DNA in the vectors herein are the prokaryote, yeast, or higher eukaryote cells. Suitable prokaryotes for this purpose include eubacteria, such as Gram-negative or Gram-positive organisms, for example, Enterobacteriaceae such as Escherichia, e.g., E. coli, Enterobacter, Erwinia, Klebsiella, Proteus, Salmonella, e.g., Salmonella typhimurium, Serratia, e.g., Serratia marcescans, and Shigella, as well as Bacilli such as B. subtilis and B. lichenifonnis, Pseudomonas such as P. aeruginosa, and Streptomyces. These examples are illustrative rather than limiting.

In addition to prokaryotes, eukaryotic microbes such as filamentous fungi or yeast are suitable expression hosts. Saccharomyces cerevisiae, or common baker's yeast, is the most commonly used among lower eukaryotic host microorganisms. However, a number of other genera, species, and strains are commonly available and useful herein, such as Schizosaccharomyces pombe; Kluyveromyces hosts such as K. lactis, K. fragilis, etc.; Pichia pastoris; Candida; Neurospora crassa; Schwanniomyces such as Schwanniomyces occidentalis; and filamentous fungi such as Penicillium, Tolypocladium, and Aspergillus hosts such as A. nidulan, and A. niger.

Numerous baculoviral strains and variants and corresponding permissive insect host cells from hosts such as Spodoptera frugiperda (caterpillar), Aedes aegypti (mosquito), Aedes albopictus (mosquito), Drosophila melanogaster (fruitfly), and Bombyx mori have been identified. A variety of viral strains for transfection are publicly available, e.g., the L-1 variant of Autographa california NPV and the Bm-5 strain of Bombyx mori NPV, and such viruses may be used as the virus herein according to the present invention, particularly for transfection of Spodoptera frugiperda cells.

Plant cell cultures of cotton, corn, potato, soybean, petunia, tomato, and tobacco can be utilized as hosts. Typically, plant cells are transfected by incubation with certain strains of the bacterium Agrobacterium tumefaciens. Regulatory and signal sequences compatible with plant cells are available, such as the nopaline synthase promoter and polyadenylation signal sequences.

Examples of useful mammalian host cell lines are mouse L cells (L-M[TK-], ATCC#CRL-2648), monkey kidney CV1 line transformed by SV40 (COS-7, ATCC CRL 1651); human embryonic kidney line (293 or 293 cells subcloned for growth in suspension culture; baby hamster kidney cells (BHK, ATCC CCL 10); Chinese hamster ovary cells/-DHFR(CHO); mouse sertoli cells (TM4); monkey kidney cells (CV1 ATCC CCL 70); African green monkey kidney cells (VERO-76, ATCC CRL-1 587); human cervical carcinoma cells (HELA, ATCC CCL 2); canine kidney cells (MDCK, ATCC CCL 34); buffalo rat liver cells (BRL 3A, ATCC CRL 1442); human lung cells (W138, ATCC CCL 75); human liver cells (Hep G2, HB 8065); mouse mammary tumor (MMT 060562, ATCC CCL51); TR1 cells; MRC 5 cells; FS4 cells; and a human hepatoma line (Hep G2).

Host cells are transfected with the expression vectors for polypeptide production, and cultured in conventional nutrient media modified as appropriate for inducing promoters, selecting transformants, or amplifying the genes encoding the desired sequences. Mammalian host cells may be cultured in a variety of media. Commercially available media such as Ham's F10 (Sigma), Minimal Essential Medium ((MEM), Sigma), RPMI 1640 (Sigma), and Dulbecco's Modified Eagle's Medium ((DMEM), Sigma) are suitable for culturing the host cells. Any of these media may be supplemented as necessary with hormones and/or other growth factors (such as insulin, transferrin, or epidermal growth factor), salts (such as sodium chloride, calcium, magnesium, and phosphate), buffers (such as HEPES), nucleosides (such as adenosine and thymidine), antibiotics, trace elements, and glucose or an equivalent energy source. Any other necessary supplements may also be included at appropriate concentrations that would be known to those skilled in the art. The culture conditions, such as temperature, pH and the like, are those previously used with the host cell selected for expression, and will be apparent to the ordinarily skilled artisan.

### Translation Kinetic Mapping

A kinetic map may be derived for a coding sequence of interest, for use in design, including without limitation harmonization, of optimized coding sequences for the source organism or for a different expression host organism. A kinetic map utilizes a translation speed index for each of the 61 codons in a given organism. In some methods of the invention, a translation speed index is derived for each codon in an organism of interest; a kinetic map of a coding sequence of interest is derived using the index at each codon; and a sequence of interest is synthesized or modified according to desired values for the kinetic at each codon. In some such methods, the desired values correspond to the kinetic map of a native coding sequence. In some such methods, the sequence of interest is operably joined to a promoter. In some such methods the sequence of interest is expressed in a host cell.

To assign a translation speed index (i) to each of the 61 codons in a given organism, the following rules can be assigned regarding the nature codon(N₁N₂N₃):anticodon(N₃₄N₃₅N₃₆) interactions (where N₁N₂N₃ represents each codon along the 5′-3′ direction in an mRNA and N₃₄N₃₅N₃₆ represents the 5→3′ anticodon loop of the decoding tRNA): (1) Watson-Crick interactions are allowed to occur between N₁N₂G₃:C₃₄N₃₅N₃₆, N₁N₂C₃:G₃₄N₃₅N₃₆, N₁N₂A₃:U₃₄N₃₅N₃₆, N₁N₂U₃:A₃₄N₃₅N₃₆ and N₁N₂C₃:I₃₄N₃₅N₃₆ (where I₃₄ represents inosine, derived from post-transcriptional deamination of some A₃₄-bearing tRNAs); (2) non-Watson-Crick interactions are allowed to occur between N₁N₂G₃:U₃₄N₃₅N₃₆, N₁N₂U₃:G₃₄N₃₅N₃₆, N₁N₂U₃:I₃₄N₃₅N₃₆, and N₁N₂A₃:I₃₄N₃₅N₃₆. Inosination is assumed to occur for all A₃₄-bearing tRNAs in eukaryotes and for A₃₄-bearing tRNAs that decode Arg codons in bacteria. Since a U₃₄A₃₅C₃₆-bearing species of tRNA is generally utilized to decode AUA codons in bacteria, it is assumed that a U₃₄A₃₅C₃₆-bearing tRNAs would partition equally for decoding AUG and AUA codons. In order to obtain normalized values for tRNA gene abundances across organisms for each codon, divide the number of tRNA genes for every codon by the total number of tRNA genes in the respective synonymous codon group.

These values (termed NNN_{%} for each codon) are then utilized, according to the above assumptions, to calculate a translation speed index (i) for each codon (termed NNNᵢ) in a given organism according to the following formulas, which translation speed index includes a weighting factor w, where w is a “penalizing” factor for non-Watson-Crick interactions.

The value for w may be empirically determined, or may be assigned, for example as a value of 3.

One approach to determining the penalizing factor for non-Watson-crick pairing is to calculate ribosome occupancy for each codon using the methods as set forth in the Examples. First, individual ribosome footprint sequences are assigned to strata defined by C-U differential. Occupancy calculations are performed independently within each stratum in order to ensure comparison among sequences of similar composition and remove the influence of composition biases.

Within each CU stratum, a basal “expected” occurrence of each codon c (expected counts_{c,u}) is calculated by taking the average occurrences of the codon in four “EPA-flanking” sites within the ribosomal footprint (the non-tRNA-interacting sites −2 and −1 to the E-site and +1 and +2 to the A-site).

The representation of each codon c in each stratum u is the ratio of the observed counts of the codon occupying a given ribosomal site, s, to the expected counts (determined from the flanking sites):

$${Representation}_{c , s , u} = \frac{{counts}_{c , s , u}}{expected {counts}_{c , u}} \qquad ( 1 )$$

The ribosome occupancy for a given codon-site combination (codon c in residing in site s) is the average of its representation across eleven CU strata u:

$${Occupancy}_{c , s} = \frac{{{•}_{u = 1}}^{11} {representation}_{c , s , u}}{11} \qquad ( 2 )$$

(1) For all bacterial codons (except those for Ile, Met and Arg): NNUᵢ=NNU_{%}+NNC_{%}/w; NNCᵢ=NNC_{%}; NNAᵢ=NNA_{%}; NNGᵢ=NNA_{%}/w. (2) For bacterial Ile: AUUᵢ=AUU_{%}+AUC_{%}/w; AUCᵢ=AUC_{%} and AUAᵢ=AUG_{%}/w*2. (3) For bacterial Met: AUGᵢ=AUG_{%}/2. (4) For bacterial Arg: treat as a eukaryotic Arg. (5) For eukaryotic two-codon groups and both similar codons of six-codon groups: NNUᵢ=NNU_{%}+NNC_{%}/w; NNCᵢ=NNC_{%}+NNU_{%}; NNAᵢ=NNA_{%}; NNGᵢ=NNG_{%}+NNA_{%}/w. (6) For eukaryotic four-codon groups, the four similar codons of six-codon groups and Ile: NNUᵢ=NNU_{%}/w+NNC_{%}/w; NNCᵢ=NNC_{%}+NNU_{%}; NNAᵢ=NNA_{%}+NNU_{%}/w; NNGᵢ=NNG_{%}+NNA_{%}/w. Once these values are obtained for the organism of interest, they are assigned to the corresponding codons of any protein coding sequence.

In one embodiment, a kinetic map starts from initiation, and from the start of the coding sequence, i values of 30 consecutive codons were added and the average value plotted at position number 15. The same operation was performed repeatedly by sliding the window of 30 values one codon position at a time, until of the coding sequence was reached. The resulting i values are plotted as a function of codon position.

To harmonize a sequence between two organisms, a kinetic map is derived for the coding sequence of the protein of interest from the source organism. On a codon by codon basis, the speed of the source organism is matched to the relative translation speed of the expression host organism.

For example, mRNAs were engineered as follows: for codons to be translated slowly, codons that lack isoacceptor tRNA genes the expression host are selected, with the exception of methionine and tryptophan. If genes for all the anticodons of a particular amino acid are present, then the codon with the least amount of available anticodon interactions at the wobble position is selected. For sequences to be translated faster, codons with the highest number of isoacceptor tRNA genes are selected. In cases were more than one codon has the highest number of tRNA genes, the codon with the highest number of available anticodon interactions at the wobble position is selected.

Where the coding sequence is modified from a native coding sequence, the modification will include a change in at least one, at least two, at least three, at least four, at least five, at least six, at least seven, at least eight, at least, nine, at least ten, at least 1%, at least 2%, at least 4%, at least 6%, at least 8%, at least 10%, at least 20%, at least 40%, at least 60%, at least 80%, even at least 90% or least 95% of the codons that are wobble pair codons.

The polypeptide that is expressed according to the above described method may be a polypeptide originating from organisms different than said host cell, i.e. a foreign polypeptide, or it may be a polypeptide of said host cell, i.e. an endogenous polypeptide with the proviso that the modified nucleotide sequence is different from the starting sequence encoding a polypeptide of substantially the same amino acid sequence. The polypeptide may also be translated in a cell-free system, where the codons are adjusted according to the tRNAs used by the organism that is a source of tRNA for the reaction mixture.

Using an mRNA that is modified by the methods of the invention, it is possible to increase the overall rate of translation, or the rate of active protein synthesis for the encoded polypeptide by at least 5%, 10%, 20%, 30%, 40%, 50%, 60%, 70%, 80%, 90% or 100%. The increased rate of translation elongation or the rate of active protein synthesis refers to a comparison of expression of the modified nucleotide sequence with expression of the starting nucleotide sequence under comparable conditions (e.g. same host cell, same vector type etc.). Alternatively the speed of translation elongation may be reduced by increasing the number of codons that rely on wobble pairing with a tRNA anticodon, where the rate of translation is reduced by at least 5%, 10%, 20%, 30%, 40%, 50%, 60%, 70%, 80%, 90% or more. It will be understood by one of skill in the art that a reduction in translation elongation rate may result in a higher rate of correctly folded or active protein being synthesized.

Where the protein is being expressed in a cell, the amount of total protein, or the amount of correctly folded or active protein, may be increased. The term “increasing the amount of at least one polypeptide in a host cell” refers to the situation that upon expressing the modified nucleotide sequences in the host cell, a higher amount of this polypeptide is produced in a host cell compared to the situation where a non-modified starting nucleotide sequence encoding for a polypeptide of substantially the same amino acid sequence and/or function is expressed in the same type of host cells under similar conditions such as e.g. comparable transfection procedures, comparable expression vectors etc.

The term “modified nucleotide sequence” for the purposes of the present invention relates to a sequence that has been modified for expression in a host cell by adjusting the sequence of the originally different non-modified/starting nucleotide sequence to account for the tRNA complement of the host cell, specifically as it relates to codons translated via wobble base-pairing. The person skilled in the art is clearly aware that modification of the starting nucleotide sequence describes the process of optimization with respect to codon usage.

In addition to the modification of existing sequences, methods are provided for the de novo synthesis of an artificial sequence. Such methods may use algorithms known in the art for codon selection, which include, inter alia, Kosakovsky et al. (2010) PLoS One. 5(7):e11230; Delport et al. (2010) PLoS One5(7):e11587; Raiford et al. (2010) IEEE/ACM Trans Comput Biol Bioinform. 7(2):238-50; Lorimer et al. (2009) BMC Biotechnol. 9:36; and Zhang and Ignatova (2009) PLoS One. 2009; 4(4):e5036, each herein specifically incorporated by reference.

In methods for de novo design of a coding sequence, the codon selection process or algorithm utilizes the difference in translation speed between the two synonymous codons, in some cases members of a wobble codon pair, to optimize the selection of codons for a desired speed and folding efficiency. In such methods, a sequence is generally designed and may be tested in a computer model, and is then used as directions for the process of chemical synthesis. In some embodiments, a polynucleotide coding sequence is provided, in which the selection of codons is adjusted for translation speed differences.

Such methods of de novo synthesis may comprise selecting a codon for each amino acid of the polypeptide of interest, wherein at least one codon is selected from a synonymous pair, wherein within the pair, a codon that has G·C or I:C pairing with a cognate anticodon in the third position has increased speed of translation relative to a codon that has G·U or I:U pairing with a cognate anticodon in the third position; and synthesizing the nucleotide sequence. The step of synthesizing the optimized nucleotide sequence may be performed in a device for automatic synthesis of nucleotide sequences which is controlled by the computer that optimizes the nucleotide sequence.

The analysis and database storage can be implemented in hardware or software, or a combination of both. In one embodiment of the invention, a machine-readable storage medium is provided, the medium comprising a data storage material encoded with machine readable data which, when using a machine programmed with instructions for using said data, is capable of displaying any of the sequence or sequence comparisons. Such data can be used for a variety of purposes, such as instructions for polynucleotide synthesis and the like. Preferably, the invention is implemented in computer programs executing on programmable computers, comprising a processor, a data storage system (including volatile and non-volatile memory and/or storage elements), at least one input device, and at least one output device. Program code is applied to input data to perform the functions described above and generate output information. The output information is applied to one or more output devices, in known fashion. The computer can be, for example, a personal computer, microcomputer, or workstation of conventional design.

Each program is preferably implemented in a high level procedural or object oriented programming language to communicate with a computer system. However, the programs can be implemented in assembly or machine language, if desired. In any case, the language can be a compiled or interpreted language. Each such computer program is preferably stored on a storage media or device (e.g., ROM or magnetic diskette) readable by a general or special purpose programmable computer, for configuring and operating the computer when the storage media or device is read by the computer to perform the procedures described herein. The system can also be considered to be implemented as a computer-readable storage medium, configured with a computer program, where the storage medium so configured causes a computer to operate in a specific and predefined manner to perform the functions described herein.

The sequences and methods for design thereof can be provided in a variety of media to facilitate their use. “Media” refers to a manufacture that contains the signature pattern information of the present invention. The databases of the present invention can be recorded on computer readable media, e.g. any medium that can be read and accessed directly by a computer. Such media include, but are not limited to: magnetic storage media, such as floppy discs, hard disc storage medium, and magnetic tape; optical storage media such as CD-ROM; electrical storage media such as RAM and ROM; and hybrids of these categories such as magnetic/optical storage media. One of skill in the art can readily appreciate how any of the presently known computer readable mediums can be used to create a manufacture comprising a recording of the present database information. “Recorded” refers to a process for storing information on computer readable medium, using any such methods as known in the art. Any convenient data storage structure can be chosen, based on the means used to access the stored information. A variety of data processor programs and formats can be used for storage, e.g. word processing text file, database format, etc.

It is to be understood that this invention is not limited to the particular methodology, protocols, cell lines, animal species or genera, constructs, and reagents described, as such may, of course, vary. It is also to be understood that the terminology used herein is for the purpose of describing particular embodiments only, and is not intended to limit the scope of the present invention, which will be limited only by the appended claims.

Unless defined otherwise, all technical and scientific terms used herein have the same meaning as commonly understood to one of ordinary skill in the art to which this invention belongs. Although any methods, devices and materials similar or equivalent to those described herein can be used in the practice or testing of the invention, the preferred methods, devices and materials are now described.

All publications mentioned herein are incorporated herein by reference for the purpose of describing and disclosing, for example, the cell lines, constructs, and methodologies that are described in the publications, which might be used in connection with the presently described invention. The publications discussed above and throughout the text are provided solely for their disclosure prior to the filing date of the present application. Nothing herein is to be construed as an admission that the inventors are not entitled to antedate such disclosure by virtue of prior invention.

The following example is put forth so as to provide those of ordinary skill in the art with a complete disclosure and description of how to make and use the subject invention, and is not intended to limit the scope of what is regarded as the invention. Efforts have been made to ensure accuracy with respect to the numbers used (e.g. amounts, temperature, concentrations, etc.) but some experimental errors and deviations should be allowed for. Unless otherwise indicated, parts are parts by weight, molecular weight is average molecular weight, temperature is in degrees centigrade; and pressure is at or near atmospheric.

As used herein the singular forms “a”, “and”, and “the” include plural referents unless the context clearly dictates otherwise. Thus, for example, reference to “a cell” includes a plurality of such cells and reference to “the protein” includes reference to one or more proteins and equivalents thereof known to those skilled in the art, and so forth. All technical and scientific terms used herein have the same meaning as commonly understood to one of ordinary skill in the art to which this invention belongs unless clearly indicated otherwise.

### EXPERIMENTAL

### Example 1

### Wobble Base-Pairing Slows In Vivo Translation Elongation in Metazoans

In the universal genetic code, most amino acids can be encoded by multiple trinucleotide codons, and the choice among available codons can influence position-specific translation elongation rates. Using sequence-based ribosome profiling, we obtained transcriptome-wide profiles of in vivo ribosome occupancy as a function of codon identity in C. elegans and human cells. Particularly striking in these profiles was a universal trend of higher ribosome occupancy for codons translated via G:U wobble base-pairing when compared to synonymous codons that pair with the same tRNA family using G:C base pairing. These data support a model in which ribosomal translocation is slowed at wobble codon positions.

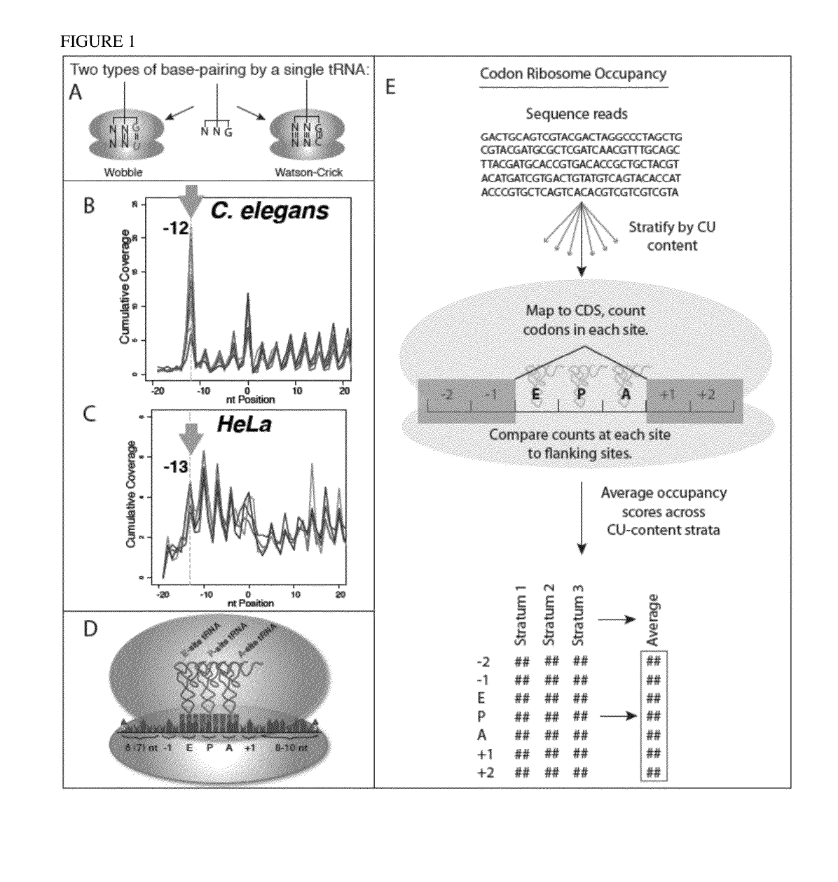

To explicitly investigate translational effects related to the character of codon:anticodon base-pairing interactions, we focused on pairs of codons that are (i) identical in the first two bases (ii) differ in the third base (cytosine in one, uracil in another), and (iii) are translated by a shared tRNA family with a G in the anticodon wobble position (FIG. 1A). These conditions, which are met for eight C/U-ending codon pairs each in C. elegans and human, allow us to compare different codon:anticodon geometries under circumstances where a single tRNA family is being used for both standard (Watson:Crick; G:C) recognition and wobble (G:U) base pairing. A second category of synonymous codon pairs (seven additional pairs in human and eight in C. elegans) are decoded by shared tRNAs derived from precursors carrying an adenine in the anticodon wobble position. This A residue is edited to inosine (Table 1) to produce a bifunctional tRNA population recognizing both C-ending and U-ending codons (I:U and I:C base pairing at the wobble position). Of the base pairs relevant to this analysis, G:C forms the highest affinity interaction, with three hydrogen bonds, I:C forms with a similar geometry but only two hydrogen bonds. G:U and I:U form with a less-favorable geometry and two hydrogen bonds.

Ingolia et al. (2009) Science 324: 218-23 recently developed a high-throughput ribosome profiling technique that utilizes the positions of mRNA fragments protected from nuclease digestion by bound ribosomes to generate a transcriptome-wide map of ribosome occupancy at sub-codon resolution. As these profiles represent a snapshot of the positions of ribosomes at a specific point in time, positions where ribosomes remain for longer time periods will have a higher probability of being captured by sequencing. The rate at which a codon is translated should thus vary inversely with the occupancy of elongating ribosomes at mRNA positions featuring this codon; i.e. the presence of a slow codon should yield increased ribosome occupancy at that specific position on an mRNA. We therefore used genome-wide ribosome profiling to probe the relationship between codon identity and ribosome occupancy and investigate codon-dependent effects on ribosome elongation speed.

### Results

We performed ribosome profiling on developmentally-synchronized C. elegans populations and on populations of cultured human cervical cancer cells (HeLa). As a reference and normalization for each sample, we sequenced matching alkali-sheared poly(A)-selected RNA with a similar size distribution (Ingolia et al. 2009). As expected if the recovered fragments were indeed ribosome footprints (and as observed by Ingolia) reads from these ribosome-protected fragments (RPFs) were enriched for lengths of ˜30-32 nt, showed a strong three-nt periodicity, and were overwhelmingly enriched in coding regions. Examining cumulative RPF distributions relative to annotated initiation codons revealed a distinctive inflection point before which few RPFs are observed and after which RPFs become abundant (FIG. 1B-C). This point is presumed to represent the location of ribosomes positioned at the initiator AUG in each message. As initiator tRNA is thought to enter at the P-site (T A Steitz 2008), this signal indicates a tentative location for the P site relative to the 5′-nt of RPFs. This identification allows us to infer the codons occupying the putative exit (E) and aminoacyl (A) sites within each RPF (FIG. 1D). The location of this signal is consistent to within +−1 nt across a range of RPF lengths, indicating that the 5′-most nt is a robust marker for determining ribosomal site location and that most length heterogeneity occurs at the 3′ end of RPFs.

In analyzing the sequence covered by RPFs from C. elegans and HeLa cells, we observe a combination of mild overall sequence composition biases (a preference for U residues at all positions) and specific codon-associated occupancy patterns. The data yield a count of the number of ribosomes captured with each codon occupying each site within the RPF (e.g., the A-site). To control for local and compositional effects (e.g., codon usage), we normalize recovered counts for each codon in a specific ribosomal site to counts across all non-tRNA-interacting sites (see METHODS for a complete description of the normalization procedure). Higher scores—indicating recovery of a given codon in a given site more often than expected—are indicative of an association between the presence of a given codon in a specific site and high ribosome occupancy. Codon-site combinations associated with slowed ribosomal elongation will thus be evident by higher scores in this assay.

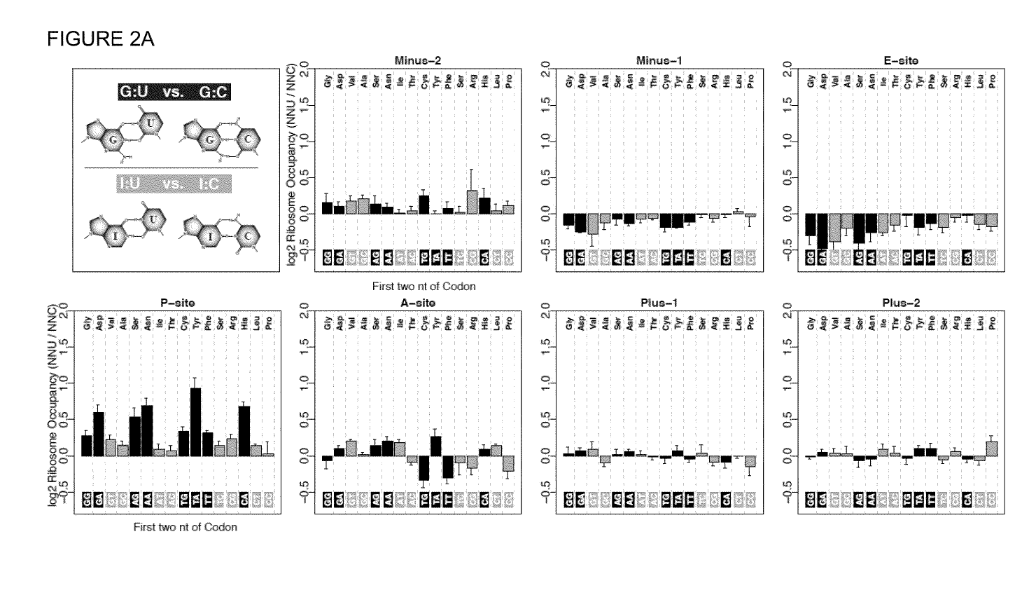

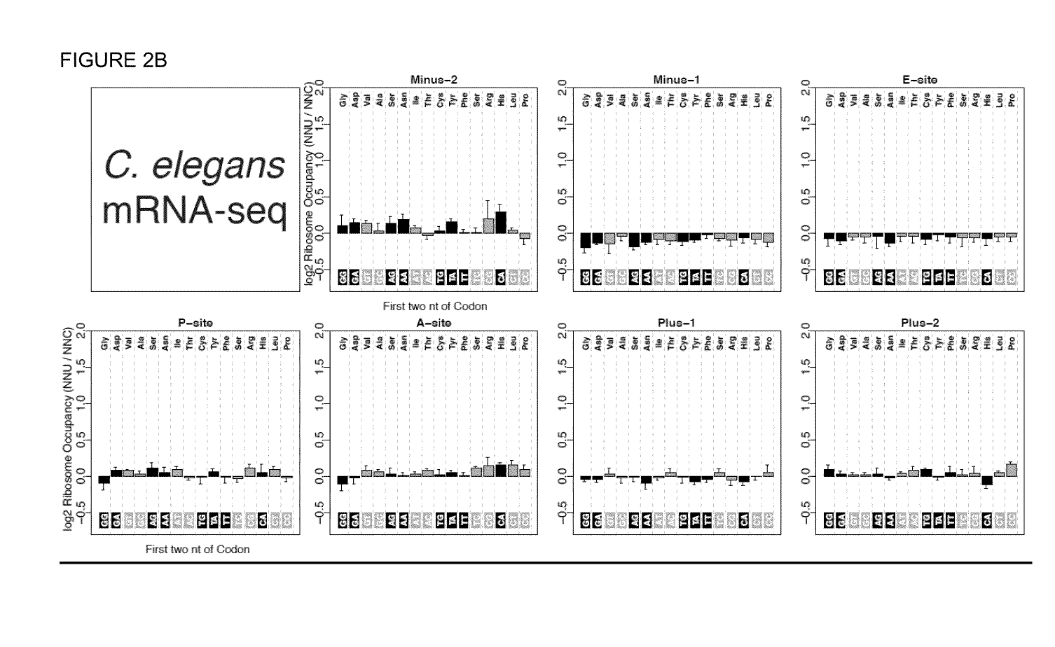

In C. elegans, examination of ribosome occupancy data for C/U-ending codon pairs reveals a striking pattern in the putative P-site position: for sixteen of sixteen NNC/NNU codon pairs, ribosome occupancy is higher for the U-ending codon (FIG. 2A). No equivalent pattern is observed in fragmented mRNA data (FIG. 2B). This difference is significantly greater among codon pairs that are read by G:C/G:U at the third position than those read by I:C/I:U. As G:U and G:C bonds differ to a greater extent in terms of geometry and bond strength (three hydrogen bonds versus two) than I:U and I:C pairs, this result suggests that the physical characteristics of base-pairing interactions at the third codon position may influence ribosome elongation.

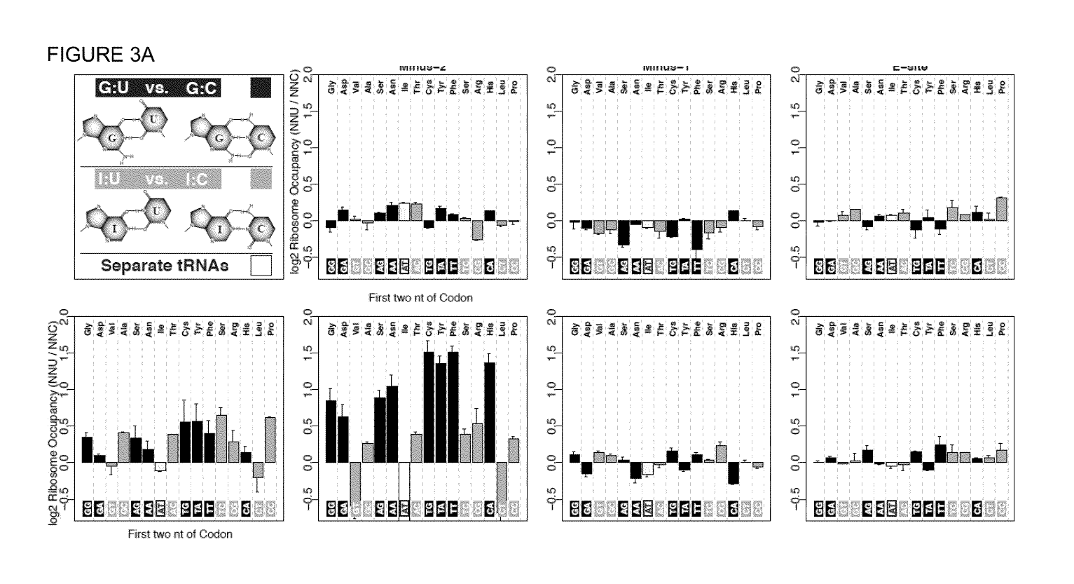

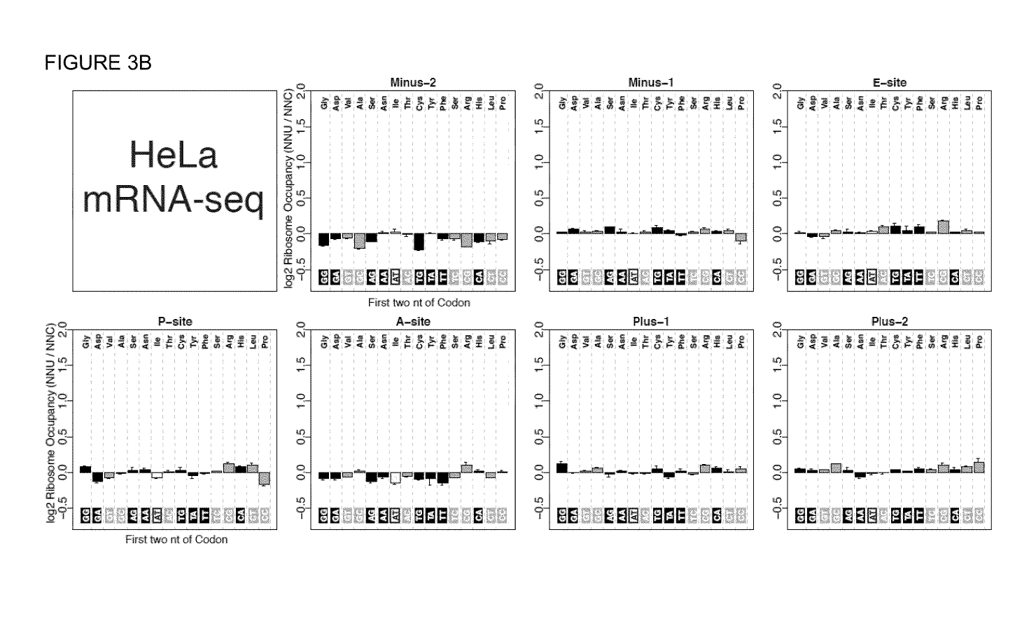

A distinctive wobble position-associated difference was also observed in HeLa cells (FIG. 3A). In ribosome profiling data from these human cells, we again observed an increase in ribosome occupancy associated with U-ending codons that is not observed for randomly-sheared mRNA (FIG. 3B). The HeLa data differed in that the effect was robust only for codon pairs translated by tRNAs using G:C/G:U pairs at the wobble position, with a weak trend evident for I:C/I:U-decoded codons. Additionally, the G:U “slowing” signal in Hela was strongest for the presumptive ribosomal A-site codon, distinct from the P-site association observed in C. elegans data. Although potentially due to a fundamental biological difference between samples, it is certainly conceivable that this single-codon shift reflects modest differences in the isolation and preparation of ribosomes and RPFs. Specifically, it is possible that during the preparation of C. elegans samples, ribosomes are able to translocate one or a few codons during tissue thawing. Single-codon precision would rely on uniformly arresting translation in two very different biological samples; ruling out a single-codon difference would be difficult with tissues from distinct sources. Despite the apparent one-codon difference, the concurrence of overall wobble-position effects between C. elegans and HeLa implicate a specific effect of base-pairing interactions on ribosome elongation rate as a potentially general metazoan phenomenon.

These data suggest a model in which wobble-position interactions could serve as significant determinants of elongation rates in the two systems. To evaluate this model, we first considered a number of technical aspects of the experimental and analytical procedures that could potentially have produced a systematic bias.

First, we compared several different experimental runs from both HeLa and C. elegans to determine whether the observed ribosome retention at wobble codons was consistently obtained. This analysis showed consistent wobble retention across two biological replicates in HeLa cells and five C. elegans samples derived from several larval stages.

Second, we considered the possibility that drug-mediated arrest of translation during RPF preparation plays a role in the observed variation in ribosome occupancy. A series of controls performed in duplicate without cycloheximide show an equivalent set of effects. These results rule out a role for this drug in the observed wobble effects.

Third, we considered the possibility that a single class of mRNA might account for the consensus effects observed. To address this, we stratified the populations of mRNA by several different criteria into distinct classes. Classification of mRNA by length and expression levels yielded wobble-position characteristics similar to those observed with the entire mRNA population in each class examined.

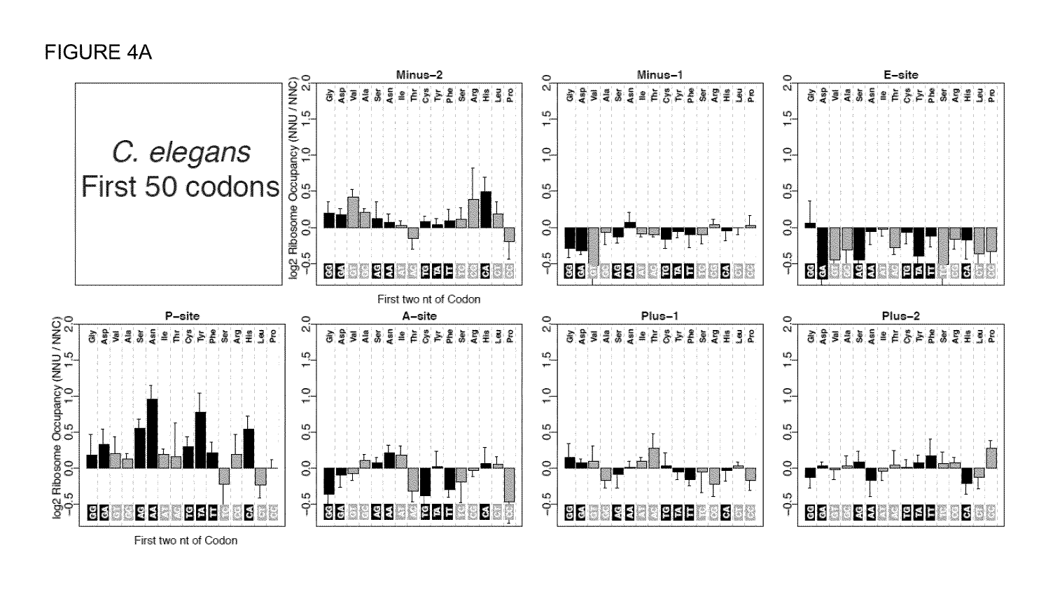

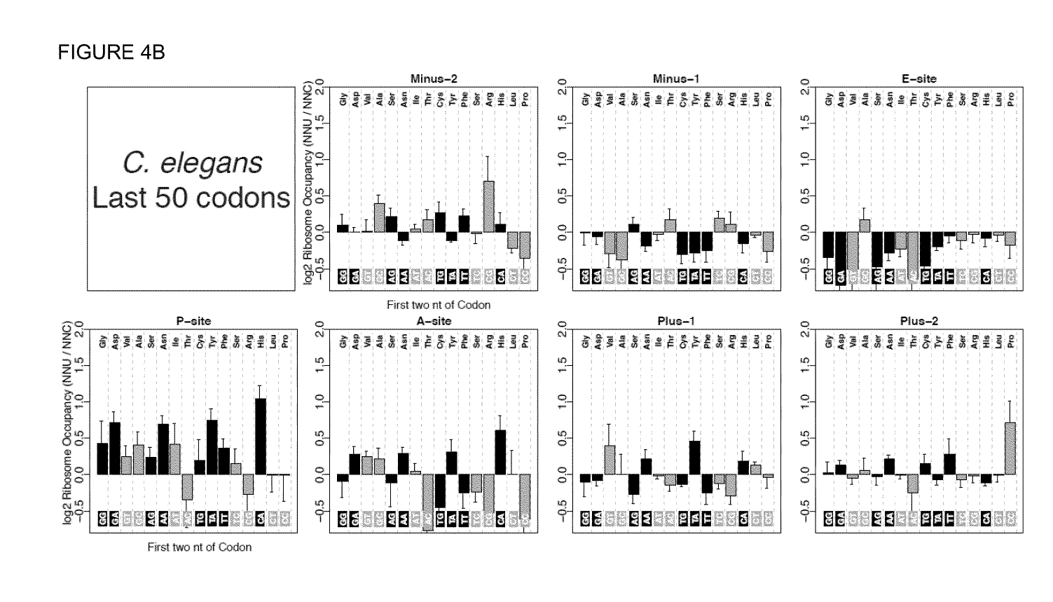

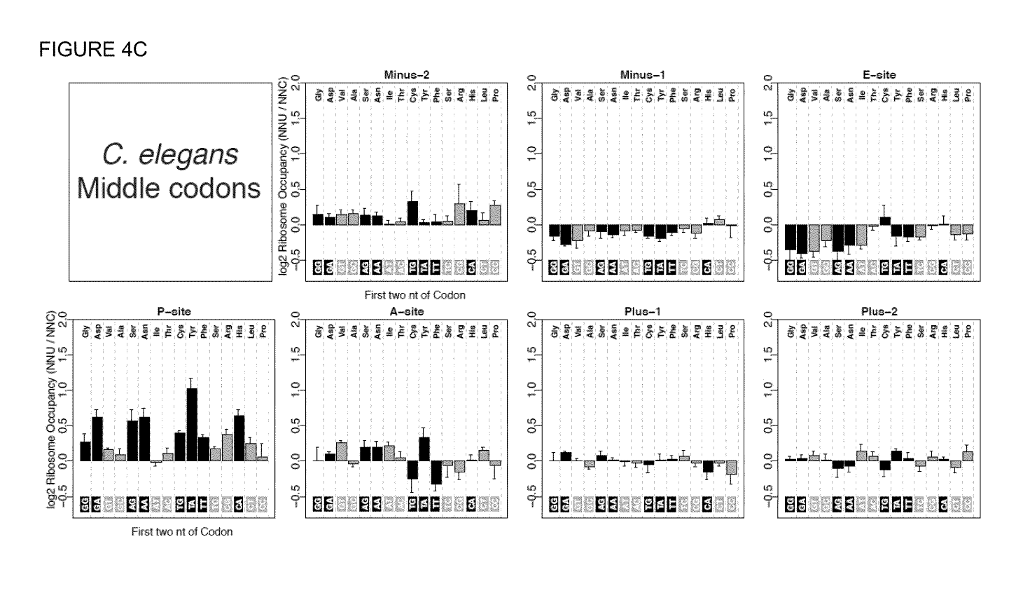

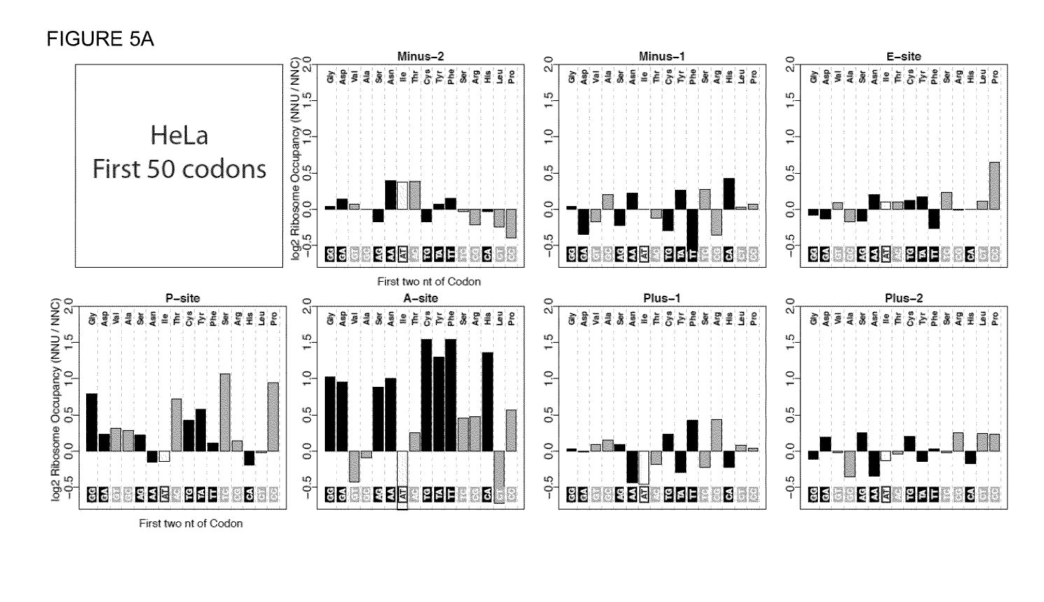

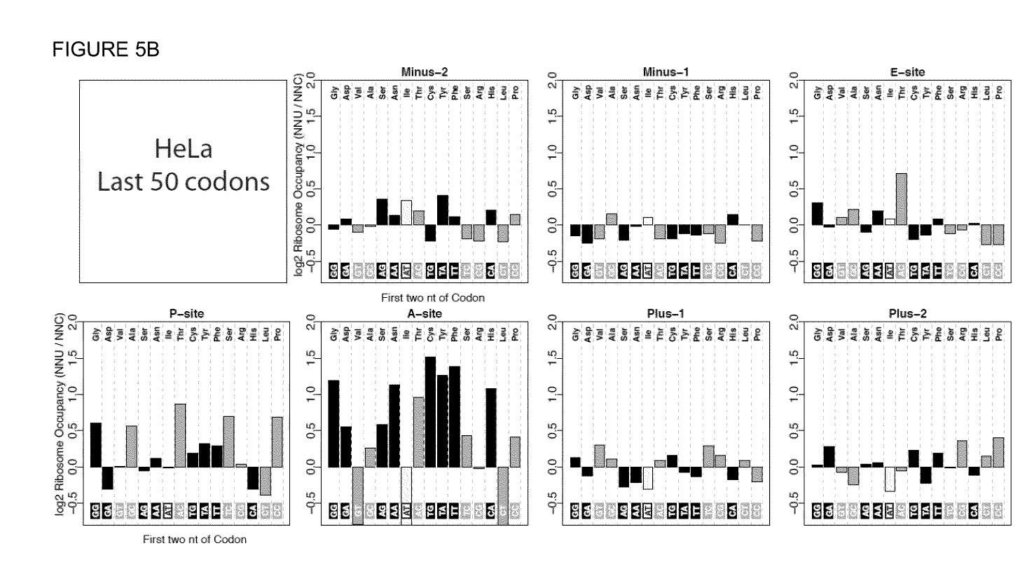

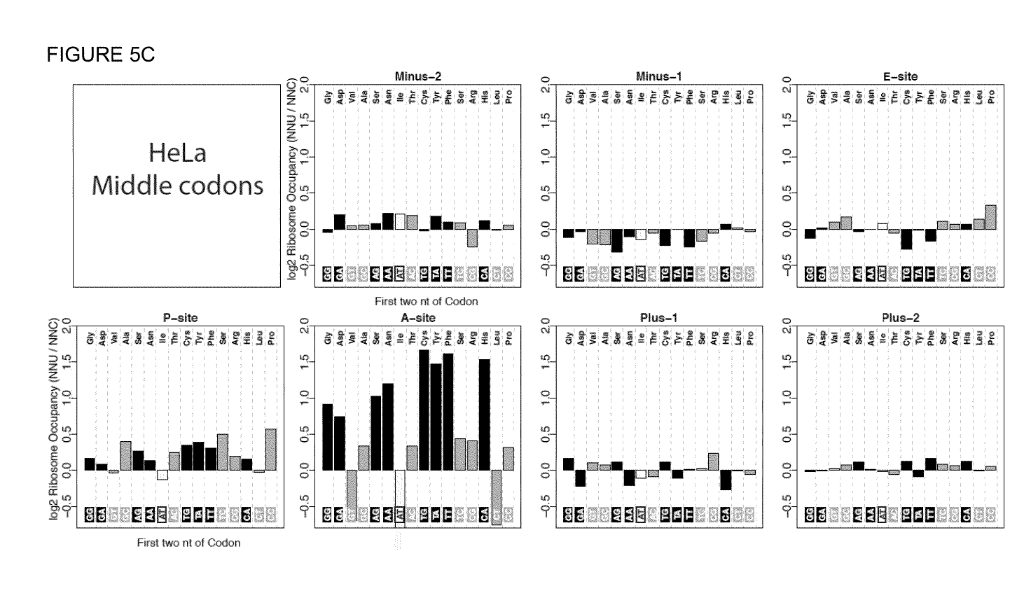

Fourth, it has been reported in some systems that ribosome occupancy and codon usage are altered at the beginning of coding sequences. Correlations between ribosome occupancy and codon usage along the length of mRNAs could account for observed occupancy patterns for wobble codons. To address this possibility, we separated mRNA sequences into early (first 50 codons in each mRNA), late (last 50 codons in each mRNA), and middle (remaining codons). This experiment revealed consistent wobble-position effects for each mRNA region (FIG. 4A-C, 5A-C). These analyses implicate the observed wobble-position effect as a consistent modulator at different stages of elongation and that it is not a result of position-specific biases in codon usage.

Fifth, we considered that wobble base-pairing could change the nuclease sensitivity of ribosome-mRNA complexes. Because we filter sequence reads by size and reading frame to examine only the highest quality RPFs, it is possible that changes in nuclease sensitivity that alter the size or reading frame of RPFs could cause reads to be excluded by our filters. To address this possibility, we repeated the analysis using all data—without filtering for reading frame or RPF length, and also repeated the analysis using only reads starting in either under-represented reading frame. In both cases, wobble-position ribosome occupancy effects were consistent with the previous analyses. We also considered that wobble-induced changes in nuclease sensitivity would result in different ribosome occupancy patterns for different sizes of RPF. We repeated ribosome occupancy analysis individually for specific RPF lengths in the range of 25 to 35 nt, observing consistent wobble-position effects within each individual length class.

Sixth, we consider the possibility that the observed wobble-related effects might reflect ribosome-independent biases in the capture and sequencing of RNA fragments. Certainly there is precedent for influences of base composition on capture, amplification, and sequencing procedures. In this case, however, it would be very unexpected to have a single internal base (in position 15 of ˜30 in C. elegans and 19 of ˜32 in HeLa) exhibit a dramatic and individualized effect on capture and sequencing. A number of sources of capture/sequencing bias are additionally ruled out by experiments in which isolated RNA was randomly sheared with alkali and subjected to the same capture and sequencing protocol (FIG. 2B, 3B).

Taken together, these assays support that the enrichment in RPF datasets for fragments containing G:U wobble codons at tRNA-binding sites represents a slowing of translation elongation at such codons, and that this effect is consistent on different classes of mRNA, for wobble codons encoding diverse amino acids, at different stages of ribosome elongation and in different eukaryotic systems. The choice of G:U versus G:C base pairing in codon:anticodon interactions represents a widely-distributed mechanism by which the kinetics of elongation can be fine-tuned.

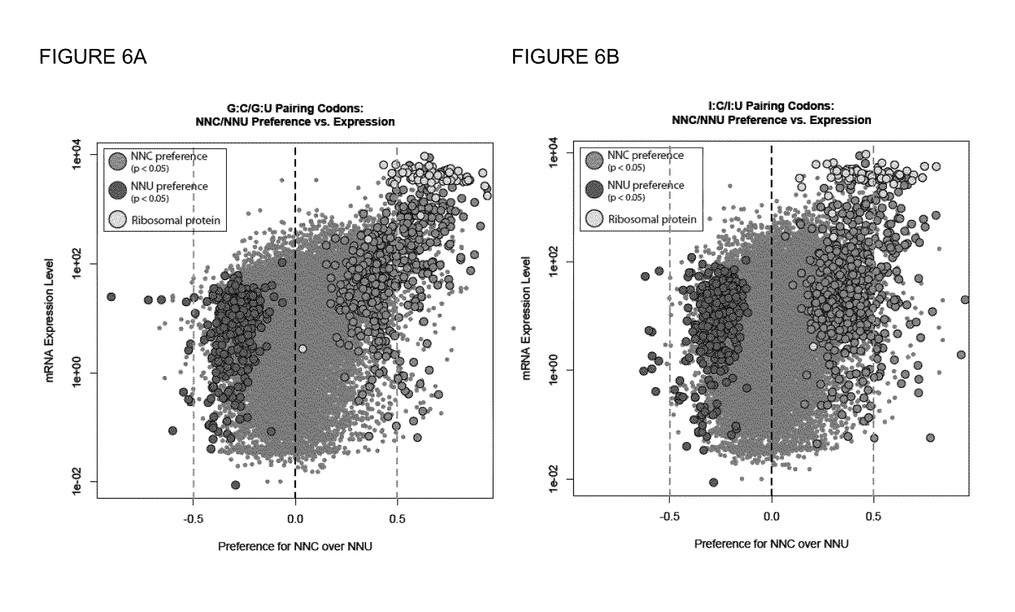

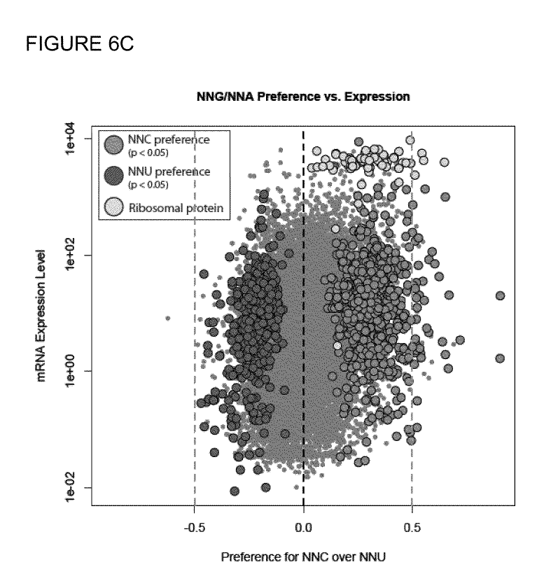

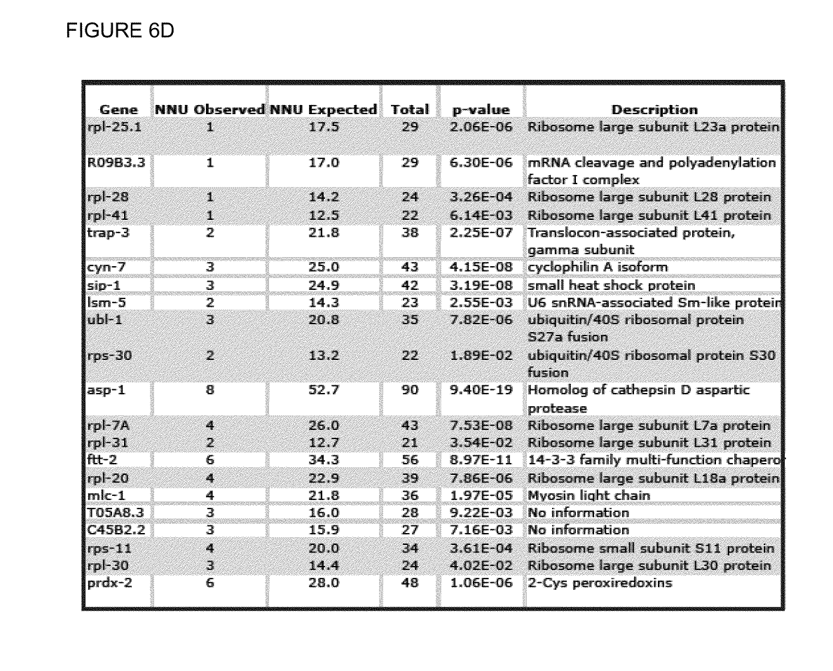

Such a mechanism might be put to different uses in distinct biological systems. To explore the possibility that “slow” versus “fast” codon choice may have specific roles in individual biological systems, we examined the relationship between codon choice and other aspects of gene activity and function. For Hela, we observe no association between gene expression levels and a preference between G:C-pairing and wobble-pairing codons. By contrast, in C. elegans, we observe that highly-expressed genes strongly favor “fast” G:C- and I:C-pairing codons over “slow” G:U- and I:U-pairing codons (FIG. 6A-B). This preference is strongest for G:C-pairing codons, mirroring the stronger effect of G:C/G:U vs. I:C/I:U pairing on ribosome occupancy. No preference of similar magnitude is observed for third-position nucleotide choice among synonymous NNG/NNA codons (FIG. 6C), nor do intronic regions of highly-expressed genes show a preference for U residues.

We expect ribosomal proteins to be one of the most reliably abundant gene classes across diverse tissues. Indeed, C. elegans ribosomal proteins are among the most highly-expressed genes and almost universally show strong preference for G:C- and I:C-pairing codons (FIG. 6A-B). Human ribosomal proteins show no such preference.

Position-specific in vivo ribosome elongation rates are certain to be influenced by a variety of factors, including availability of cognate tRNAs, mRNA secondary structure, or the presence of RNA binding proteins. We have here demonstrated one salient feature associated with modulating translation elongation rates: a ˜15% to 2-fold increase in ribosome occupancy at positions where G:U base-pairs are present in the ribosomal decoding center in C. elegans (Table S2), and a ˜65% to nearly 3-fold increase in HeLa cells (Table 3). These effects are within the normal timescale of translation, but sufficient in magnitude to change the temporal landscape of protein synthesis.

A general ability of wobble codons to slow translational elongation has interesting mechanistic, regulatory, and evolutionary implications.

On a mechanistic level, increased ribosome occupancy at a codon position on an mRNA could reflect inefficient aa-tRNA selection (the tRNA being slow to enter the site), slowed translocation (tRNA is slow to leave), or a combination of both. These influences could be focal, affecting only a single chemical step in translation, or could be distributed among several different steps in the mechanism. Although ribosomal footprinting provides remarkable precision in viewing an in vivo process, this precision is not sufficient to determine the individual step(s) affected by wobble pairing. In particular, the difference that we observe between the two species regarding the site of increased wobble occupancy (the P-site in C. elegans and the A-site in HeLa) may be due to differences in experimental parameters (e.g., a technical difference in kinetics of ribosome freezing in C. elegans sufficient to allow transit of an additional codon), or could reflect differences in the two organisms concerning which biochemical step of translation is retarded by the presence of wobble base-pairs. Discerning the mechanistic details of wobble-induced ribosome slowing is an intriguing topic for future investigation.

At a regulatory/evolutionary level, changes in the temporal landscape of translation could exert a functional influence on biological output in the form of stably and correctly folded protein. Coding sequence adjustments could modulate translational rates, and hence the initial landscapes of protein folding. One such means would be to invoke codons whose translation is limited by paucity of the corresponding tRNA, resulting in translational slowing as the machinery searches for a rare tRNA, however specific tRNA levels would need to be maintained at limiting levels to provide consistent effects on translational rate. As an alternative coding mechanism for modulating translational elongation, wobble-dependent slowing effects would allow fine-tuning of rates in a manner that does not rely maintenance of borderline tRNA pools. Such a mechanism would allow critical translation rate modulations to be “hard-coded” into mRNA sequences in a protein-specific way that would be robust to diverse developmental and metabolic conditions.

In addition to the implications for natural regulation of protein synthesis kinetics and quality, the results provided herein demonstrate that specific wobble/non-wobble codon choices may influence the biological activity of synthetic genes and genetic constructs used in biotechnology and gene therapy. Indeed optimization of kinetic characteristics—which might require faster or slower peptide synthesis for individual proteins, domains, or residues—could be among the many determinants of effective synthetic biotechnology.

### Materials and Methods

Tissue: C. elegans N2 animals were cultured as described in Brenner, 1974 (Brenner 1974). HeLa cell monolayers were incubated at 37° C. in 10% serum-supplemented Dulbecco modified Eagle medium and washed in phosphate buffered saline. Both HeLa and C. elegans samples were harvested by freezing in liquid nitrogen; these frozen pellets served as the starting material for subsequent manipulations.

Ribosome profiling and sequencing were performed using a protocol very similar to that described by Ingolia (2009), with minor modifications described in supplementary methods. Sequencing was performed on an Illumina GAII/GAIIx.

Bioinformatics: Bioinformatic analyses were carried out using custom Peri and R scripts unless otherwise noted. Sequence reads were mapped using Bowtie v.0.12.2 (Langmead et al. 2009) to the ENSEMBL GRCh37 hg19 gene track for H. sapiens and the Wormbase WS190 reference set for C. elegans. In our data analysis, we have used a variety of “stratification” approaches in which subsets of genes or codons are analyzed separately to determine if the different subsets share an observed molecular trend.

Occupancy measurement: To calculate ribosome occupancy for each codon, we stratified RPF sequences by CU content. First, individual ribosome footprint sequences were assigned to strata defined by C-U differential. Strata with difference values from −5 to 5 were used in this analysis, with analysis carried out in parallel on non-stratified samples giving comparable results. Occupancy calculations were performed independently within each stratum (described below) in order to ensure comparison among sequences of similar composition and remove the influence of composition biases.

Within each CU stratum, we determined a basal “expected” occurrence of each codon c (expected counts_{c,u}) by taking the average occurrences of the codon in four “EPA-flanking” sites within the ribosomal footprint (the non-tRNA-interacting sites −2 and −1 to the E-site and +1 and +2 to the A-site).

The representation of each codon c in each stratum u is the ratio of the observed counts of the codon occupying a given ribosomal site, s, to the expected counts (determined from the flanking sites):

$${Representation}_{c , s , u} = \frac{{counts}_{c , s , u}}{expected {counts}_{c , u}} \qquad ( 1 )$$

The ribosome occupancy for a given codon-site combination (codon c in residing in site s) is the average of its representation across eleven CU strata u:

$${Occupancy}_{c , s} = \frac{{{•}_{u = 1}}^{11} {representation}_{c , s , u}}{11} \qquad ( 2 )$$

Gene-specific wobble preference: To assess a gene's preference for using wobble-position G:C vs. G:U base-pairs, we first calculated the probability of encountering a G:C for each synonymous C/U-ending codon pair, p, based on the frequency of each codon in the set of all annotated coding sequences for the organism:

$${P ( G \text{:} C )}_{p} = \frac{{Counts}_{NNC , all CDS}}{{Counts}_{NNC , all CDS} + {Counts}_{NNU , all CDS}} \qquad ( 3 )$$

From these probabilities, we calculated an expected number of G:C codons expected for each gene, g, based on the number of occurrences of each synonymous C/U-ending codon pair (either NNC or NNU) and the probability of encountering the G:C codon calculated for each pair in eq. 5:

$$Expected ( G \text{:} C ) = {•}_{p • all G \text{:} C / G \text{:} U pairs} {Counts}_{ping} • {P ( G \text{:} C )}_{p} \qquad ( 4 )$$

A gene's preference for G:C vs. G:U wobble pairing is determined by:

$$Preference ( G \text{:} C to G \text{:} U ) = \frac{{Observed}_{G \text{:} C} - {Expected}_{G \text{:} C}}{{Expected}_{G \text{:} C}} \qquad ( 5 )$$

By comparing the number of observed G:C-pairing codons found in a gene's coding sequence to the number expected, we can examine the preference of each gene for G:C or G:U wobble pairings in a manner that accounts for the relative codon usages of each synonymous pair. An identical analysis was conducted using the set of I:C/I:U-pairing codons.

### Materials and Methods

### Samples

A. C. elegans samples. C. elegans N2 animals were cultured as described in Brenner, 1974 (Brenner 1974). Embryos from mixed stage populations were prepared by treatment with sodium hydroxide and sodium hypochlorite solution as previously described (Girard et al. 2007). Embryos were placed on NGM plates in the absence of food for 24 hours at 16° C., producing synchronized populations of L1 stage larvae. L1s were transferred to enriched agar plates with E. coli OP50 food source for: 4 hours (early L1), 34 hours (L2), 45 hours (L3), or 63 hours (L4). Animals were harvested by washing 3-4 times with 15 mL 50 mM NaCl to remove bacteria, weighing concentrated pellet, and flash-freezing aliquots in liquid nitrogen. Pellets of 0.4 or 0.2 g were stored at −80 C.

B. HeLa cells. HeLa cell monolayers were incubated at 37° C. in 10% serum-supplemented Dulbecco modified Eagle medium. Cells were harvested by washing with phosphate buffered saline, pelleting, and freezing in liquid nitrogen.

### Ribosome Profiling and Library Preparation

Profiling and library preparation were performed largely as described in Ingolia et al, 2009. Because of multiple minor alterations, we report the full protocol below:

A. Nuclease digest and sedimentation. Flash freezing was used as the initial means to arrest ribosomes. UV traces taken after sucrose sedimentation confirm that higher-order polysomes remain intact after freezing (FIG. S1M). Frozen tissue pellets were crushed in the presence of liquid nitrogen with a mortar and pestle, and resulting powder was transferred to an excess of ice cold polysome lysis/digest buffer (20 mM Tris pH 8.0, 140 mM KCl, 1.5 mM MgCl₂, 1% Triton, 100 μg/mL cycloheximide [left out in cycloheximide-free experiments]). Lysate was homogenized by vortexing ˜30 seconds, then transferred to a fresh 1.5 mL tube and brought to 650 μL with lysis buffer. RNase I (Ambion #AM2295) was added to lysate using concentrations determined empirically for each sample by titration of RNAse I (Ingolia et al. 2009) followed by examination of sucrose gradient traces after digestion (500 U to 0.4 g C. elegans, 40 U to 0.1 g C. elegans, 130-160 U to ˜0.1-0.15 g HeLa pellet). After mixing by inversion, tubes were placed at 23° C. for one hour. The RNasel reaction was stopped by the addition of 100 μL of 100 mg/mL heparin sulfate (Sigma #H-3393).

Experiments were performed with two batches of RNase I. The first batch was used to calibrate the original C. elegans samples and then to calibrate HeLa digests at a later date. In subsequent C. elegans experiments, we observed a decrease in enzyme activity and thus switched to a second, fresh enzyme batch for later C. elegans experiments. The enzyme batch used is explicitly noted in table S4. Lysates were pipetted onto 10-50% sucrose gradients (0.3M NaCl, 15 mM MgCl₂, 15 mM TrisHCl pH 7.5, 0.1 mg/ml cycloheximide [left out in cycloheximide-free experiments]) and centrifuged in an SW-41 rotor at 35,000 rpm for three hours and ten minutes. Gradients were fractionated by reverse pumping with 60% sucrose. ˜700 μL fractions were frozen at −80° C.; 80S ribosome-containing fractions were identified from the UV trace and pooled for RNA purification.

Total RNA was isolated from sucrose fractions by hot acid-phenol extraction. A 650 μL sucrose fraction was thawed rapidly at 65° C., followed by the addition of 1/10^{th} volume 10× proteinase K buffer (500 mM NaCl, 100 mM Tris pH 7.4, 50 mM EDTA), 4 μL 15 mg/mL glycoblue (Ambion #AM9516), 20 μL 20% SDS, and 4 μL 20 mg/mL proteinase K (Roche #3115879). The mixture was incubated at 65° C. for one hour. The protease-treated fraction was then extracted twice with one volume of acid-phenol chloroform (Ambion #AM9720), once with one volume of chloroform, and then precipitated with sodium acetate and >3 volumes ethanol at −80° C.

B. poly(A)+ RNA isolation and fragmentation. Poly(A)+ RNA was isolated from frozen worm/cell pellets using the Ambion Micropoly(A) Purist kit (Ambion #AM1919) frozen tissue protocol. Resulting RNA was mixed with an equal volume of alkali RNA fragmentation solution (2 mM EDTA, 100 mM Na₂CO₃ pH 9.2) and incubated at 94° C. for 40 minutes, followed by quenching and ethanol precipitation (Ingolia et al. 2009).

C. Library Preparation. RNA samples were treated with T4 Polynucleotide kinase (NEB #M0201S) (12.5 μL RNA in H₂O, 1.5 μL 10×PNK buffer, 1 μL PNK) for 90 minutes at 37° C. Reaction product was loaded on a 12% PAGE-urea gel (National Diagnostics #EC-833) with a 30 nt ssDNA marker oligo (5-CATGTAAGAGGTTGGTCTCCTTTGGCGGGA-3) and a 10 bp DNA ladder (Invitrogen #10821-015). RNA of size ˜24-36 nt was excised, physically disrupted with a pipette tip, and eluted overnight at 4° C. with gentle rotation in 0.3 M NaCl and 50 U Superase-in (Ambion #AM2696). Size-selected fragments were poly-adenylated using E. coli poly(A) polymerase (NEB #MO276S) (11 μL RNA, 1.5 μL 10×PAP buffer, 1.5 μL 10 mM ATP, 0.5 μL Superase-in, 0.5 μL PAP) for 30 minutes at 37° C., followed by quenching with 80 μL 5 mM EDTA and ethanol precipitation. Poly(A) RNA was resuspended in 12 μL RNasefree H₂O. 1 μL 10 mM dNTPs and 0.5 μL 100 μM ONTI225/AF-MS-40 (/5Phos/GATCGTCGGACTGTAGAACTCTGAACCTGTCGGTGGTCGCCGTATCATT/iSp18/CACTCA/iSp18/CAAGCAGAAGACGGCATACGATTTTTTTTTTTTTTTTTTTTVN) were added and the mixture incubated at 65° C. for five minutes, followed by addition of 4 μL 5×FSB, 0.5 μL Superase-in, 1 μL 0.1M DTT, and 1 μL Superscript III reverse transcriptase (Invitrogen #18080-093). RT reaction was incubated at 48° C. for forty minutes, and RNA hydrolyzed by the addition of 2.3 μL 1M NaOH and a twelve minute incubation at 98° C. The RT product was mixed with 22.3 μL Ambion GLB2 (Ambion #AM8547) and loaded onto a 12% PAGE-urea gel, run at 900V for at least three hours. Reverse transcription product was cut away from unincorporated RT primer, and the gel slice physically disrupted and eluted overnight with gentle agitation at 4° C. in 0.3 M NaCl. Eluent was ethanol precipitated and resuspended in 15 μL sterile H₂O. RT product was circularized with circligase (Epicentre #CL4111K) (15 μL cDNA, 2 μL 10× circligase buffer, 1 μL 50 mM MnCl₂, 1 μL 1 mM ATP, 1 μL circligase) at 60° C. for one hour. Either one or two μL of circularization reaction was used in a 30 μL PCR reaction (6 μL HF buffer, 0.6 μL 10 mM dNTPs, 0.3 μL 100 μM AF-MS-22 (5-CAAGCAGAAGACGGCATACG-3), 0.3 μL100 μM AF-MS-43 (5-AATGATACGGCGACCACCGACAGGTTCAGAGTTCTACAGTCCGACG-3), 0.3 μL Phusion polymerase (NEB #F-530S), H₂O to 30 μL). Amplification was carried out with a 30 second initial denaturation at 98° C., followed by 7-21 cycles of 98° C. for 10 s, 60° C. for 10 s, and 72° C. for 10 s. Amplified products were separated on a 3% NuSieve (Cambrex #50080) agarose gel and purified with a Qiagen QiaQuick gel extraction kit (Qiagen #28706). Samples were sequenced on an Illumina GAII/GAIIx.

### Sequence Analysis.

Bioinformatic analyses were carried out using custom Peri and R scripts unless otherwise noted.

A. Initial read processing and mapping. Solexa reads were trimmed of all contiguous trailing A residues (introduced during library preparation by poly(A) polymerase) and resulting reads longer than 20 nt were used for mapping. Reference sequences for mappings consisted of the total set of predicted/verified coding sequences for the respective organisms, using the ENSEMBL GRCh37 hg19 gene track for H. sapiens and the Wormbase WS190 (Birney et al. 2004) reference set for C. elegans. For transcripts with multiple isoforms, a single isoform was selected arbitrarily, and the resulting sequence set was filtered to remove sequences that contained perfect overlapping 24-mers with another transcript. Reads were mapped to these reference sets using Bowtie v.0.12.2 (Harris et al. 2010) reporting all reads that were perfect matches or contained a single mismatch to the reference.

B. Identifying ribosomal sites within RPFs. To establish a relationship between codons and ribosome occupancy, it was necessary to determine, given the position of an RPF read, which codons were occupying which positions within the ribosome. Ribosomes contain three tRNA binding sites: the exit (E), peptidyl (P), and aminoacyl (A) sites. To determine the position of each of these sites relative to the 5′ end of an RPF, we examined the positions of RPF starts relative to the initiation codon. Reference sequence sets were generated from the C. elegans and human transcriptomes featuring coding sequences plus 20 nt of 5′ UTR sequence. Reads from all RPF sequencing sets were mapped to these references using bowtie as described in III-A. The positions of the first nucleotide of all RPFs on all transcripts were used to generate a cumulative coverage score at each nt position relative to the start codon. The cumulative coverage calculation normalizes inputs on a per transcript basis such that each transcript contributes equally to the overall signal:

$$Cumulative coverage , position j = {\sum }_{i ∈ iall transcripts} \frac{Reads mapping at position j of transcript i}{Total reads mapping to transcript i}$$

Plotting cumulative coverage revealed a strong signal for reads that begin 12 nt upstream of the start codon in C. elegans, and a corresponding signal at −13 nt in HeLa cells (FIG. 1B-C). As very few reads mapped upstream of these positions, this signal provides a snapshot of ribosomes positioned at the start of translation.

Stratifying this analysis by RPF size (i.e., executing the analysis separately on each size class of RPF in a range from 24-35 nt) shows a consistent signal to +/−1 nt in each species, relative to the 5′ ends of RPFs (FIG. S1K-L). This suggests that most length heterogeneity occurs at the 3′ end, making distance from the 5′ end a robust landmark for mapping ribosomal sites across the range of RPF sizes. Given that the initiator methionyl tRNA occupies the P-site during 80S ribosome formation (Langmead et al. 2009), we infer that canonical RPFs consist of 12 and 13 nt 5′ to the P-site in C. elegans and HeLa cells, respectively. Ingolia at al. determined a similar structure for yeast RPFs, with 12 nt 5′ to the P-site. These features allow provisional positioning of E, P, and A-sites within each individual RPF (FIG. 1D).

C. Calculating ribosome occupancy for ribosomal sites. Before calculating ribosome occupancy, sequencing reads were filtered based on length and reading frame. For standard calculations (FIGS. 2, 3), only reads with a length greater than 26 nt and beginning in the species-canonical reading frame were included in an effort to select the highest quality RPFs for analysis. For control purposes, various alternative filter parameters were used in parallel analysis, including no filter (FIG. S4A, S5A), filters selecting a single nt length (FIG. S4D, S5D), and filters for reads starting in the other two reading frames (FIG. S4B-C, S5B-C). All non-standard filter parameters are explicitly described in the text and figure legends where appropriate. In all cases where parallel calculations are shown, the same filter parameters were applied to both RPF and fragmented mRNA data.

U residues are over-represented in a position-independent manner in RPF data. Fragmented mRNA controls rule out a U bias deriving from capture and sequencing of RNA fragments. U-rich sequences are therefore preferentially recovered from the digest, sedimentation, or extraction procedure, a fact that may represent a technical feature of the experiments or a feature of ribosome biology.

To avoid any systematic effects of base composition, we used two approaches 1) performing comparisons between sequences of similar CU content, and 2) comparing codon occupancy between positions located within the ribosome footprint. The latter approach controls for region or mRNA-specific biases in nucleotide composition, as the same population of mRNA segments is sampled.

Occupancy Measurement:

To calculate ribosome occupancy for each codon, we stratified RPF sequences by CU content. First, individual ribosome footprint sequences were assigned to strata defined by C-U differential. Strata with difference values from −5 to 5 were used in this analysis, with analysis carried out in parallel on non-stratified samples having comparable results. Occupancy calculations were performed independently within each stratum (described below) in order to ensure comparison among sequences of similar composition and remove the influence of composition biases. Within each CU stratum, we determined a basal “expected” occurrence of each codon c (expected counts_{c,u}) by taking the average occurrences of the codon in four “EPA-flanking” sites within the ribosomal footprint (the non-tRNA-interacting sites −2 and −1 to the E-site and +1 and +2 to the A-site).

The representation of each codon c in each stratum u is the ratio of the observed counts of the codon occupying a given ribosomal site, s, to the expected counts (determined from the flanking sites):

$${Representation}_{c , s , u} = \frac{{counts}_{c , s , u}}{expected {counts}_{c , u}} \qquad ( 1 )$$

The ribosome occupancy for a given codon-site combination (codon c in residing in site s) is the average of its representation across eleven CU strata u:

$${Occupancy}_{c , s} = \frac{{{\sum }_{u = 1}}^{11} {representation}_{c , s , u}}{11} \qquad ( 2 )$$

To investigate the effectiveness of stratification-based data normalization, we have examined the robustness of observed wobble codon effects (a) at different stages in C. elegans development (FIG. S2A-E), (b) at different sites within the mRNA (first 50 codons, last 50 codons, intervening areas; FIG. 4, 5), (c) at different expression levels (as assessed by counts of mRNA tags in our sheared mRNA capture; FIG. S2L-N, S31-K), (d) for different mRNA lengths (FIG. S2I-K, S3F-H), (e) for different RPF lengths (FIG. S4D, S5D), (f) in different C-U strata (or without C-U stratification; FIG. S4E, S5E). The observed wobble codon effects were robust across all of these stratifications.

D. mRNA abundance from mRNA-seq data. mRNA abundance measurements (FIGS. 6, S2L-N, S3I-K, S6) were calculated as the reads per kilobase of mRNA per million reads mapping to the reference (RPKM). For C. elegans, RPKM values were derived from the combination of all mRNA-seq experiments at four larval stages, providing a general assessment of average mRNA abundance that is not specific to any particular developmental stage or tissue. For HeLa, RPKM values were derived from two biological replicates of cultured cells.

E. Wobble codon preference. The set of all protein coding sequences (CDS) from wormbase release WS190 and from ENSEMBL hg19 GRCh37 were used for analysis of wobble codon preference. Sequences were not filtered to remove mRNAs sharing sequence identity with other transcripts, as was done for ribosome occupancy analysis. To assess a gene's preference for using wobble-position G:C vs. G:U base-pairs, we first calculated the probability of encountering a G:C for each synonymous C/U ending codon pair, p, based on the frequency of each codon in the set of all annotated coding sequences for the organism:

$${P ( G \text{:} C )}_{p} = \frac{{Counts}_{NNC , all CDS}}{{Counts}_{NNC , all CDS} + {Counts}_{NNU , all CDS}} \qquad ( 3 )$$

From these probabilities, we calculated an expected number of G:C codons expected for each gene, g, based on the number of occurrences of each synonymous C/U-ending codon pair (either NNC or NNU) and the probability of encountering the G:C codon calculated for each pair in eq. 5:

$$Expected ( G \text{:} C ) = {\sum }_{p ∈ all G \text{:} C / G \text{:} U pairs} {Counts}_{ping} \cdot {P ( G \text{:} C )}_{p} \qquad ( 4 )$$

A gene's preference for G:C vs. G:U wobble pairing is determined:

$$Preference ( G \text{:} C to G \text{:} U ) = \frac{{Observed}_{G \text{:} C} - {Expected}_{G \text{:} C}}{{Expected}_{G \text{:} C}} \qquad ( 5 )$$

By comparing the number of observed G:C-pairing codons found in a gene's coding sequence to the number expected, we can examine the preference of each gene for G:C or G:U wobble pairings in a manner that accounts for the relative codon usages of each synonymous pair. An identical analysis was conducted using the set of I:C/I:U pairing codons.

P-values for wobble nucleotide preference were calculated using binomial tests with Bonferroni correction representing the probability of obtaining the number of observed NNC codons by random sampling given the total number of relevant codons pairs (trials) and the expected number of G:C-pairing codons. Calculations for NNA/NNG preferences were performed in the same manner as described above. All codon frequencies were taken from the UCSC genome database (PP Chan and Lowe 2008).

The table in FIG. 6D represents genes with the most extreme preferences for NNC. First genes were identified whose preference is statistically significant with a Bonferonni corrected p<0.05. Genes were then sorted by the magnitude of their NNC preference.

F. Detecting editing of wobble-position tRNA nucleotides. Total RNA was isolated from C. elegans N2 L4 stage animals and frozen HeLa cells using the Mirvana (Ambion AM1560) total RNA isolation protocol. RNA was sheared by alkali hydrolysis and prepared for Illumina sequencing using the circligase method as described in (II). RNAseq reads were mapped to C. elegans or H. sapiens tRNA reference sequences from the UCSC genomic tRNA database (PP Chan and Lowe 2008) using Bowtie v.0.12.2, reporting all reads with two or fewer mismatches. The average read that aligned to at least one tRNA aligned to 1.13 and 1.19 tRNA species (all tRNAs sharing an anticodon) for C. elegans and HeLa cells, respectively, and all mappings were kept. For assessing editing, the base at the first anticodon position for all reads aligning to a tRNA was queried. Reverse transcriptase incorporates C opposite inosines, so A-to-I editing is evidenced by the detection of a G residue where the genomic sequence is an A.

TABLE 1A
A-to-I editing at the first base of the tRNA anticodon in C. elegans.

| | | Wobble nt | | | | |
|---|---|---|---|---|---|---|
| Anticodon | UCSC gene | A | G (I) | U | C | % A−>I |
| AGC | trna79-AlaAGC | 33 | 3989 | 46 | 6 | 97.9% |
| AAU | trna261-IleAAT | 39 | 4587 | 9 | 2 | 98.9% |
| AAG | trna239-LeuAAG | 11 | 3560 | 3 | 3 | 99.5% |
| AGU | trna2-ThrAGT | 15 | 5358 | 77 | 4 | 98.2% |
| AGA | trna262-SerAGA | 24 | 3329 | 39 | 0 | 98.1% |
| AAC | trna57-ValAAC | 203 | 6099 | 189 | 47 | 93.3% |
| AGG | trna29-ProAGG | 65 | 880 | 28 | 26 | 88.1% |
| ACG | trna82-ArgACG | 14 | 415 | 1 | 0 | 96.5% |

* I residues are read as G by reverse transcriptase.

TABLE 1B
A-to-I editing at the first base of
the tRNA anticodon in HeLa cells.

| | | Wobble nt | | | | |
|---|---|---|---|---|---|---|
| Anticodon | UCSC gene | A | G (I) | U | C | % A−>I |
| ACG | trna8-ArgACG | 66 | 1362 | 6 | 0 | 95.0% |
| GTT | trna47-AsnGTT | 0 | 2708 | 19 | 0 | 99.3% |
| AAG | trna3-LeuAAG | 21 | 620 | 0 | 0 | 96.7% |
| AGT | trna4-ThrAGT | 19 | 2279 | 12 | 0 | 98.7% |
| AAT | trna80-IleAAT | 3 | 681 | 3 | 1 | 99.0% |
| AGG | trna11-ProAGG | 113 | 3507 | 16 | 20 | 95.9% |
| AGC | trna108-AlaAGC | 22 | 53 | 0 | 0 | 70.7% |
| AGA | trna147-SerAGA | 10 | 240 | 0 | 1 | 95.6% |

* I residues are read as G by reverse transcriptase.

TABLE 2
Ribosome Occupancy Data (log2) for NNC/NNU codons in C. elegans.

| | | | Fold |
|---|---|---|---|
| | | | Occupancy |
| Codon | Occupancy | Occupancy | Difference |
| Stem | NNU | NNC | (NNU/NNC) |
| G:C/G:U-pairing | | | |
| GG | 0.238 | −0.038 | 1.21 |
| GA | 0.919 | 0.323 | 1.51 |
| AG | 0.394 | −0.137 | 1.44 |
| AA | 0.456 | −0.232 | 1.61 |
| UG | 0.368 | 0.032 | 1.26 |
| UA | 0.350 | −0.580 | 1.91 |
| UU | −0.019 | −0.337 | 1.25 |
| CA | 0.004 | −0.673 | 1.60 |
| I:C/I:U-pairing | | | |
| GU | −0.024 | −0.249 | 1.17 |
| GC | −0.058 | −0.205 | 1.11 |
| AU | −0.667 | −0.758 | 1.07 |
| AC | 0.128 | 0.062 | 1.05 |
| UC | −0.568 | −0.706 | 1.10 |
| CG | −0.009 | −0.246 | 1.18 |
| CU | −0.336 | −0.533 | 1.11 |
| CC | 0.696 | 0.665 | 1.02 |

TABLE 3
Ribosome Occupancy Data (log2) for NNC/NNU codons in HeLa.

| | | | Fold |
|---|---|---|---|
| | | | Occupancy |
| Codon | Occupancy | Occupancy | Difference |
| Stem | NNU | NNC | (NNU/NNC) |
| G:C/G:U-pairing | | | |
| GG | 0.247 | −0.605 | 1.81 |
| GA | −0.302 | −0.926 | 1.54 |
| AG | 0.059 | −0.824 | 1.84 |
| AA | −0.301 | −1.345 | 2.06 |
| UG | 0.476 | −1.041 | 2.86 |
| UA | 1.228 | −0.130 | 2.56 |
| UU | 1.016 | −0.449 | 2.86 |
| CA | 0.574 | −0.790 | 2.57 |
| I:C/I:U-pairing | | | |
| GU | −1.144 | −0.556 | 0.67 |
| GC | −0.319 | −0.584 | 1.20 |
| AC | 0.123 | −0.269 | 1.31 |
| UC | 0.430 | 0.038 | 1.31 |
| CG | −0.299 | −0.838 | 1.45 |
| CU | −0.065 | 0.619 | 0.62 |
| CC | 0.247 | −0.076 | 1.25 |

### Example 2

### Elongation Rates and Folding Efficiencies of Nascent Polypeptides can be Predictably Manipulated by Engineering Synonymous Codon Substitutions Along their Coding Sequence

We have developed a metric to predict organism-specific polypeptide elongation rates of any mRNA based on whether each codon is decoded by tRNAs capable of Watson-Crick, non-Watson-Crick or both types of interactions. We demonstrate by pulse-chase analyses in living E. coli cells that sequence engineering based on these concepts predictably modulate translation rates due to changes in polypeptide elongation and show that such alterations significantly impact the folding of proteins of eukaryotic origin. Finally, we demonstrate that adjustment of codon choices based on expression host tRNA pools designed to mimic natural ribosome movement of the original organism can significantly increase the folding efficiency of the encoded polypeptide. To describe the matching of translation rates between source and host cells, Angov et al. (2008) make use of the term “harmonization” of translational elongation rates in describing their approach in matching source and host codon frequencies to engineer E. coli production of proteins encoded by the highly AT-rich plasmidium genome. Although Angov et al. used a different experimental basis, different elongation rate surrogates, and distinct computational procedures in their choices of codons for recoding than are used in the studies described here, the overall approaches share the goal of structure-based tuning of translation rates toward optimizing protein yield, and we likewise adopt the generic term “Harmonization” to refer to this kind of attempted optimization.

Here, we utilize pulse-chase methods to measure in vivo translation rates of mRNA recoded based on the two most common predictors of translation speed: tRNA concentration and codon bias. We show that the former is the superior translation speed predictor because it accelerates translation rate beyond that of the codon bias recoded mRNA. Furthermore, rate acceleration decreases the folding efficiency of the recoded gene products. This prompted us to construct an mRNA where codons were chosen based on matching the natural variations in speed (observed in our translation speed profiles) along the mRNA sequence with a metric based on tRNA pools in the host, E. coli, which resulted in increased folding efficiency of the recoded gene product.

### Results

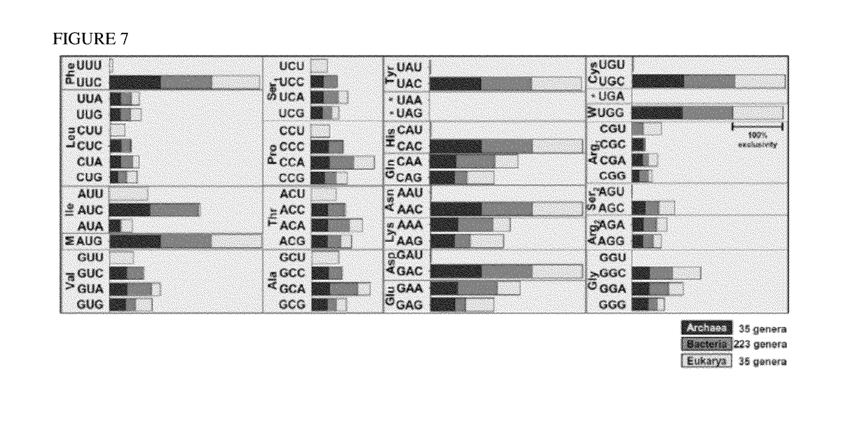

Patterns of tRNA gene content differ significantly among the three domains of life. To gain insight into the extent by which different organisms utilize different sets of tRNAs during protein synthesis, we conducted an analysis of the current version of the Genomic tRNA Database (GtRNAdb)₁₂, a manually curated database documenting the predicted number of genes for each tRNA isoacceptor for a large number of organisms whose genomes have been sequenced. Upon examination of organism-specific tRNA gene content for all genera available in the database (35 archaea, 223 bacteria and 35 eukaryotes), we observed striking differences in the pattern of tRNA genes present in the genomes of organisms belonging to each domain of life (FIG. 7). The distribution of tRNA genes for most synonymous codons within a Domain tends to be rather constant, but clear differences arise when comparisons are made across the three Domains. For instance, in the case of isoleucine (encoded by AUU, AUC and AUA), most bacteria have tRNA genes that decode AUC, and none that decode AUU. In eukaryotes, the situation is reversed: most have tRNA genes for AUU and only very few have genes for AUC (only a small fraction of organisms in all three domains have tRNA genes that decode AUA). In other cases, the tRNA gene is present in a considerable fraction of eukaryotic genomes, yet completely absent in bacterial genomes (for example, GUU, CCU, CUU, UCU, ACU and GCU). In yet other cases, all three domains appear to contain mostly the same tRNA genes for a particular isoacceptor, especially for amino acids with only two synonymous codons (for example, UAC, CAC, AAC, GAC and UGC), but even in these cases, a minor fraction of eukaryotic genomes may contain “rare tRNA genes” that decode the other isoacceptor (UAU, CAU, AAU, GAU and UGU), whereas no bacterial genomes appear to possess them.

It is important to emphasize at this point that absence of tRNA genes that decode a particular codon in a given organism is not correlated with the absence (or underrepresentation) of that codon in the protein coding sequences of that organism. In other words, all cellular organisms utilize all 61 codons to encode proteins, even though certain tRNA genes are missing in every genome analyzed to date. In fact, we have observed in our analyses that, in every organism, some of the most frequent codons have no matching tRNA isoacceptor genes. When such a codon for which there is no tRNA isoacceptor gene(s) is encountered by the ribosome, it is decoded by a tRNA that base pairs to it via non-Watson-Crick interactions (i.e., by wobble). For certain codons, it has been shown experimentally that such non-Watson-Crick codon-anticodon interactions result in decreased elongation rates compared to decoding via strict Watson-Crick binding.

Since both eukaryotes and bacteria lack tRNAs for a significant number of codons (FIG. 7) (and the abundances of those present vary substantially), it is likely that the non-uniform movement of the ribosome along an mRNA will be considerably influenced by the pool of available tRNAs in each organism. Thus, it may be possible that, although bacterial ribosomes are entirely capable of decoding the genetic information of eukaryotes, their different patterns of tRNA availability may lead to differences in the rates at which various segments of the polypeptide emerge from the ribosome. Such variations in the rates of appearance of segments of the polypeptide that are critical for folding may contribute to the often observed misfolding of eukaryotic proteins upon production in bacteria. For example, a subtle increase in the concentration of a partially folded intermediate during translation of its polypeptide sequence may exceed the critical concentration of the intermediate and lead to its nucleation-dependent aggregation, thus forming intracellular aggregates. In order to explore these differences, we sought to develop a formula that would allow us to predict the relative polypeptide elongation rates along a given mRNA on any expression host whose genome has been annotated with respect to tRNA gene content.

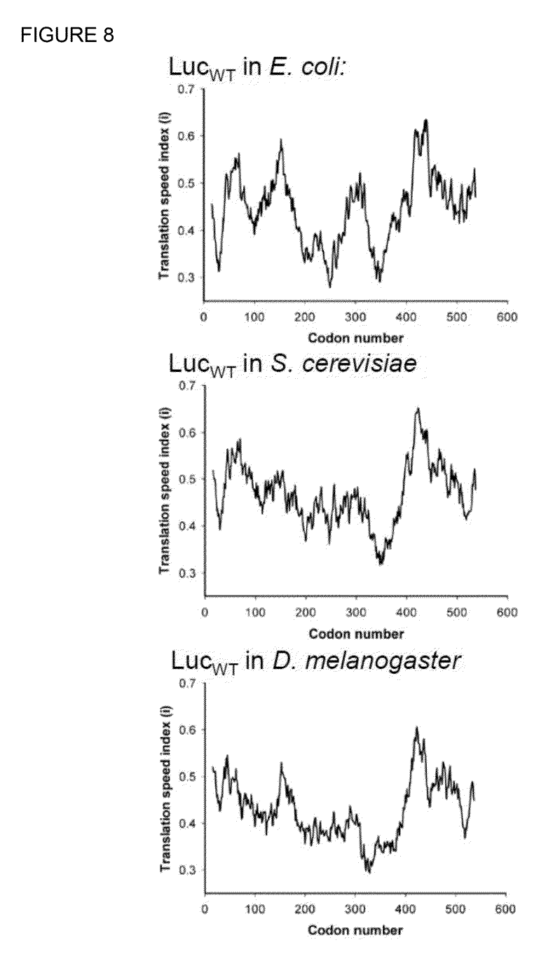

Prediction of relative polypeptide elongation rates based on expression host tRNA availability. We wished to develop a metric to assess the influence of the different patterns of tRNA availability of bacteria and eukaryotes on the relative rates of emergence of the nascent polypeptide. Our metric (see Materials and methods) generates a relative speed value for each codon along an mRNA molecule based on whether the cognate Watson-Crick tRNA isoacceptor is present for that codon in the expression host, whether non-Watson-Crick tRNA isoacceptors capable of decoding that codon are present as well as the number of tRNA genes that fulfill one and/or both of the above conditions. As mentioned previously, it has been shown that codons differing only in the wobble position are translated by the same tRNA species at different rates. We utilized the experimentally determined translation rates of 31 individual codons and current knowledge of the tRNAs responsible for decoding them to calculate a general ratio of wobble-based decoding to Watson-Crick-based decoding (see Materials and methods). The relative speed values thus obtained for each codon are then averaged over a sliding window of 30 codons (which corresponds to the number of amino acid residues the ribosomal exit tunnel can accommodate). These values, which we have termed “translation speed index” are plotted against codon position to generate a profile that depicts the predicted variations in polypeptide elongation rates based on tRNA availability of a given expression host (FIG. 8). Regions of high relative speed value (or translation speed index) predict a faster polypeptide elongation rate compared to regions of lower translation speed value. Due to the similarities in tRNA gene content among eukaryotes, such profiles should be similar for a eukaryotic coding sequence translated by another eukaryote but different when translated by a bacterium. When we examine the sequence that encodes the enzyme luciferase from the firefly Photinus pyralis, a model protein whose folding behavior has been previously characterized in our laboratory we see that the predicted differential translation speed profiles are indeed similar between an insect (D. melanogaster) and a yeast (S. cerevisiae), but different in the bacterium E. coli (FIG. 8). Accordingly, we have found that luciferase folds well when recombinantly produced in yeast, but not in bacteria. Interestingly, the predicted region of fastest translation speed for luciferase in the eukaryotic profiles correlates well with the presence the C-terminal domain (residues 437-544), a topologically independent structural domain of the enzyme.

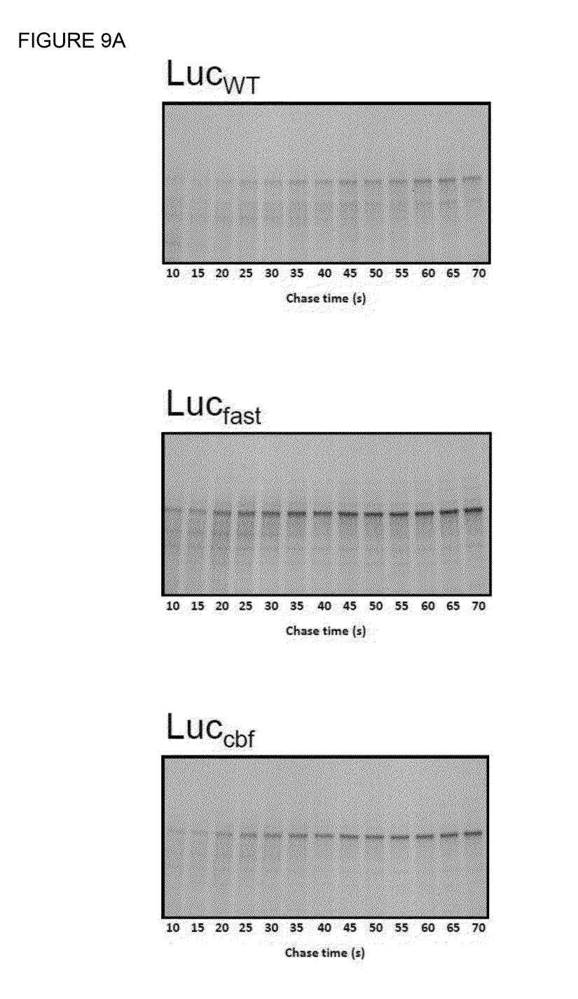

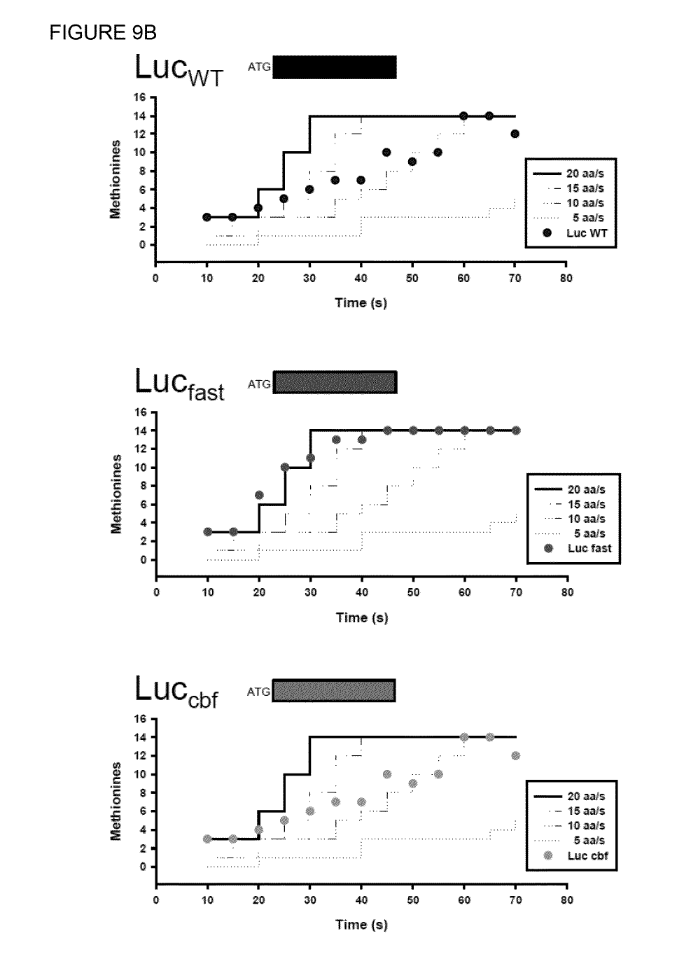

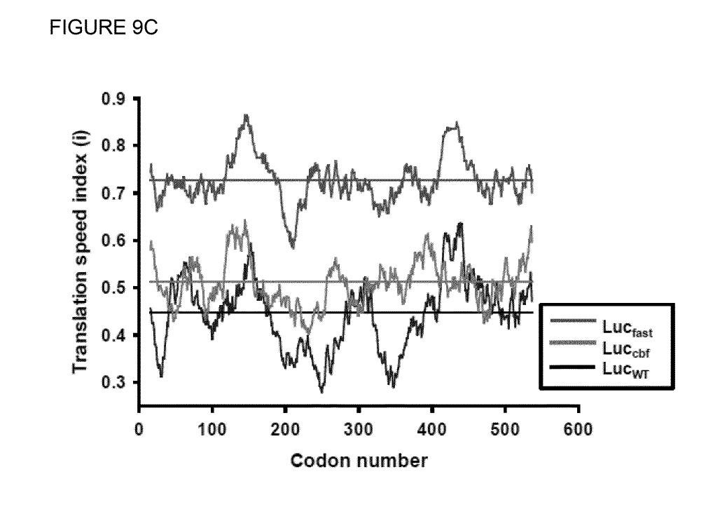

Polypeptide elongation rates can be predictably accelerated by manipulating wobble base composition. We next sought to determine whether the predicted different rates by which individual codons are decoded (depending on whether they are read by tRNAs capable of binding via Watson-Crick vs. non-Watson-Crick interactions) were of sufficient magnitude to affect overall ribosome movement along an mRNA molecule in vivo. We began by asking whether a sequence whose codons were decoded exclusively by tRNAs pairing via Watson-Crick interactions would be translated at observably faster rates than the original wild type sequence. In both cases, the actual amino acid sequences emerging from the ribosome are identical. We employed DNA synthesis to build a bacterial expression construct for the model protein firefly luciferase in which every amino acid is encoded by a synonymous codon read by the tRNA species with the highest number of tRNA genes in E. coli. This synthetic construct (termed Luc_{fast}) and the original luciferase sequence (Luc_{WT}) were placed under control of identical regulatory sequences (T7-driven promoter and terminator) for expression in E. coli and their respective mRNAs accumulated to similar levels. We then proceeded to determine their polypeptide elongation rates by performing pulse-chase analyses in live E. coli cells (Materials and methods). We found that, indeed, luciferase protein synthesis was clearly accelerated in cells harboring the Luc_{fast} construct compared to those harboring the Luc_{WT} plasmid (FIG. 9a). In order to obtain a quantitative idea of the magnitude of the observed rate acceleration, we generated simulated curves of the calculated rates of appearance of methionine incorporated into full length firefly luciferase at various predicted average polypeptide elongation rates₂ (FIG. 9b) (Materials and methods). As can be observed, the rate of full length protein appearance from Luc_{WT} most closely fits the theoretical curve corresponding to 10 amino acids per second (aa/s), whereas that produced from Luc_{fast} clearly approaches 20 aa/s. Indeed, least-squares analysis revealed that the best fit for the Luc_{WT} data corresponded to a translation rate of 9.8 aa/s.

Previously, predictions of the speed at which codons are translated have been based on their frequency of occurrence in a given set of coding sequences in a given organism. In this so-called biased codon usage (or codon bias), frequent codons have traditionally been considered fast, while rare ones have been predicted to be translated more slowly. We next considered whether sequence engineering based on this metric would also lead to rate acceleration. We designed a luciferase sequence composed entirely of the most frequently used codons in E. coli, regardless of the number of tRNA genes associated with those codons (termed Luc_{cbf}) (references and GtRNAdb). Pulse-chase analysis revealed that protein production rates from the LUC_{cbf} plasmid was intermediate between those of Luc_{fast} and Luc_{WT} (FIG. 9a). Since a considerable fraction of codons predicted to be translated fast by codon usage bias criteria correspond to the codons for which the highest number of tRNA genes exist in E. coli, it is not surprising that the luciferase produced from Luc_{cbf} accumulated with faster rates than that produced from Luc_{WT} and probably occurred as a result of the over-representation of those codons. Indeed, predictions based on our metric suggested that Luc_{cbf} would be translated with rates intermediate between those of Luc_{WT} and Luc_{fast} (FIG. 9c).

Next, we wished to assess whether we could employ a reasoning similar to the above to engineer a sequence that would be translated more slowly. Thus, we synthesized a luciferase construct composed of codons relying solely on non-Watson-Crick decoding tRNAs for their translation (except for codons encoding Met, Trp and Gln), which we termed Luc_{slow}. This construct was placed under regulatory sequences identical to those described above. Although we could detect luciferase activity in cells harboring this plasmid, we could not accurately measure accumulation of full length protein in our pulse-chase analyses, precluding determination of polypeptide elongation rates for protein produced from this construct. It is probable that such pronounced frequency of codons relying on wobble tRNA interactions for decoding led to marked ribosomal stalling, which resulted in sequestration of ribosomes and the consequent activation of cellular mechanisms to rescue such ribosomes, leading to very little production of full length protein. Regardless, we believe that our results, taken together, suggest that predictions based on tRNA availability (based for example on the presence and number of tRNA genes) rather than biased codon usage might yield more accurate results regarding the translation rates of synonymous codons, at least in E. coli.

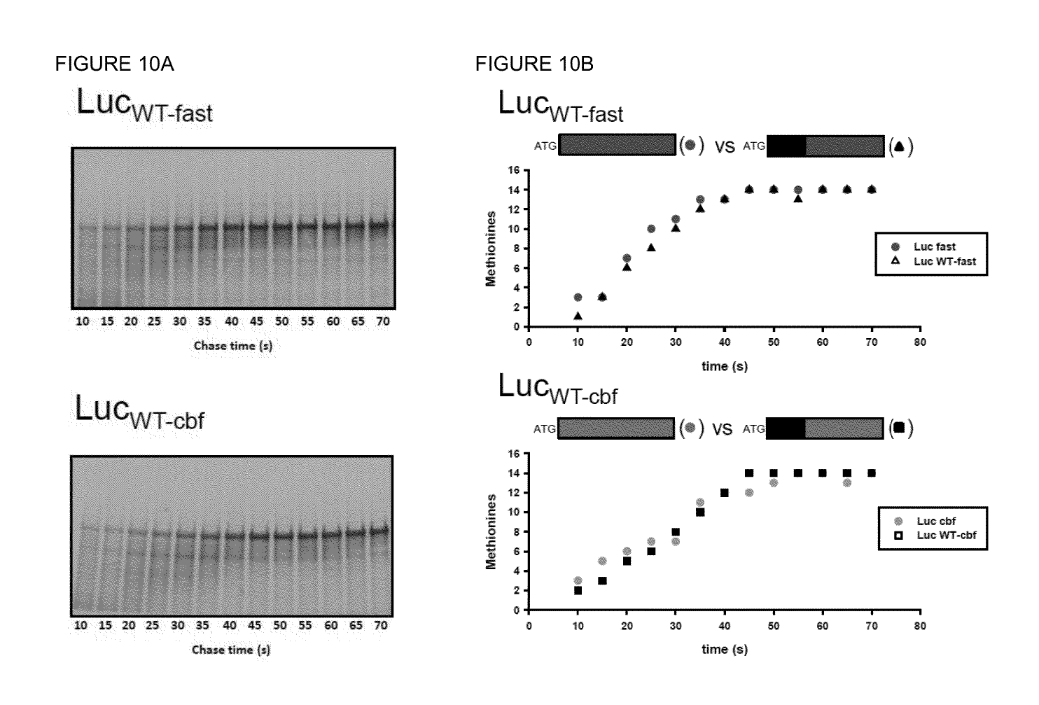

Translation initiation does not play a significant role in sequence-based acceleration. It is well known that nucleotide composition can influence mRNA secondary structure and that secondary structural elements in regions at and/or near the ribosomal binding and translation initiation sites can significantly affect translation initiation rates. Although all our constructs contained identical ribosomal binding sites and their mRNA stabilities around critical translation initiation sites were similar, we nevertheless wished to ensure that changes in translation initiation were not responsible for our observed effects on translation acceleration. We engineered a set of sequences in which the first 50 codons were identical among themselves (derived from the Luc_{WT} sequence, to yield Luc_{WT-fast} and Luc_{WT-cbf}) and conducted experiments in the same manner as before. As can be observed (FIG. 10b), Luc_{WT-fast} and the Luc_{WT-cbf} lead to production of full length proteins with accelerated rates similar to those of their Luc_{fast} and Luc_{cbf} counterparts (FIG. 9a). Thus, the presence of wild type translation initiation sites does not affect the overall effects on rate acceleration conferred to by the rest of the sequences. We believe that changes in mRNA secondary structure throughout the sequence are unlikely to mediate the observed effects, as the Luc_{fast} construct actually contains a higher GC content (54%) than Luc_{WT} (45%). Thus, even though Luc_{fast} might contain more stable secondary structural elements (which could provide an impediment to ribosomal movement, we nevertheless observe a significant rate acceleration, which argues for the robustness of this sequence manipulation and suggests it is due primarily to an effect on polypeptide elongation.

Acceleration of translation rates by synonymous codon substitutions impacts the folding of the encoded polypeptide. We have previously utilized E. coli strains harboring mutant ribosomes that can translate at variable rates, depending whether the antibiotic streptomycin is absent (˜5aa/s) or present (˜11 aa/s) to investigate the effects of translation rates on protein folding efficiencies₁₅. We have shown that the folding efficiency of firefly luciferase (and several other recombinant proteins of eukaryotic origin) increases about two-fold when produced by ribosomes translating at slower rates. Our coding sequence-based manipulations described above now allowed us to test whether further increases in polypeptide elongation rates beyond those observed under wild type conditions had an impact on folding efficiencies.

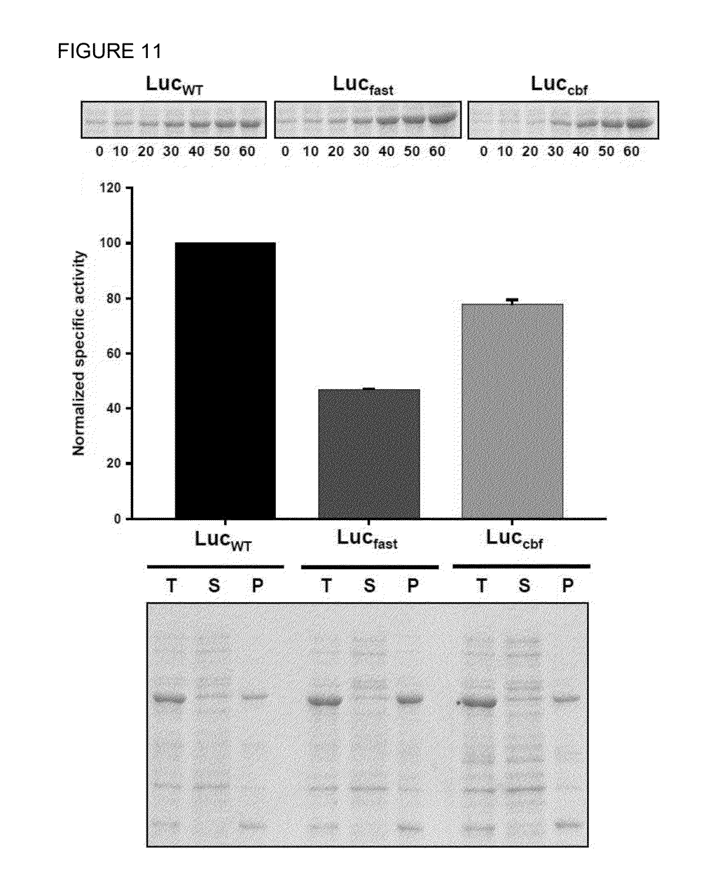

To elucidate the effect of rate acceleration on folding efficiency, we expressed our set of sequence-engineered luciferase constructs, determined the accumulation total (folded and misfolded), soluble (presumably folded) and aggregated (misfolded) recombinant protein produced, and measured luciferase activity as an indication of acquisition of the native state (FIG. 11) (Materials and methods). It is important to note here that the amino acid sequences among all these sequence-engineered constructs are predicted to be identical, as all manipulations involved synonymous substitutions. Consistent with our pulse-chase results, the steady-state accumulation of Luc_{fast} is faster and reaches higher levels than Luc_{WT}, and Luc_{cbf} displays a behavior intermediate between Luc_{fast} and Luc_{WT}. As can be observed, at similar levels of total recombinant protein accumulation, the protein produced from the Luc_{fast} construct is present predominantly in aggregated (pellet) fraction, while Luc_{WT} resulted in a greater proportion of recombinant protein in the soluble fraction at the expense of aggregated material (FIG. 11). Most importantly, when luciferase activities were measured, it was clear that ribosomal acceleration resulted in 2-fold decrease in folding efficiency. Consistent with the results presented above, the luciferase translated from the Luc_{cbf} construct exhibited an intermediate degree of solubility and specific activity. Thus, it appears that, at least for firefly luciferase, increments in overall polypeptide elongation rates correlate with decrements in folding efficiency, consistent with our previously published results.

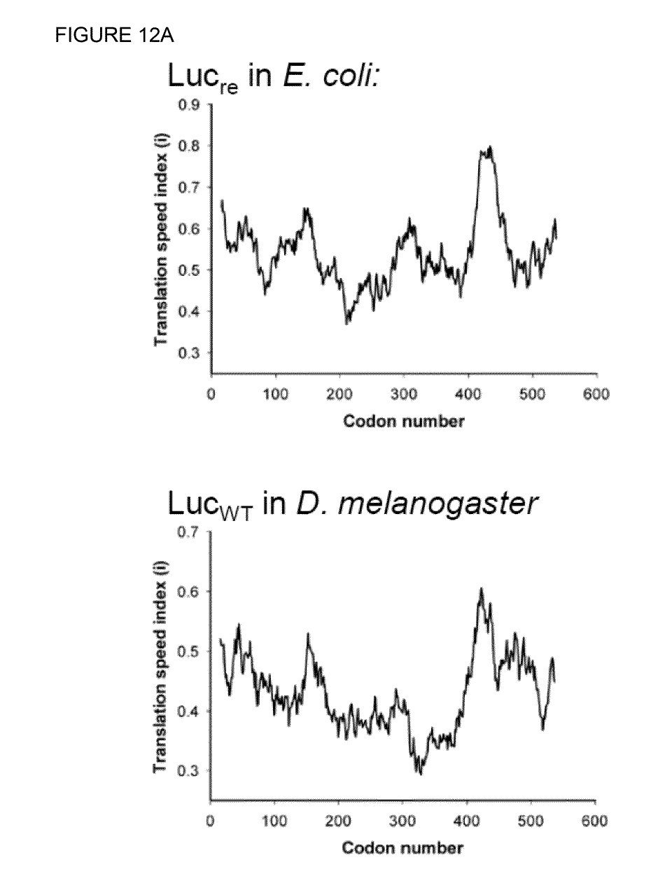

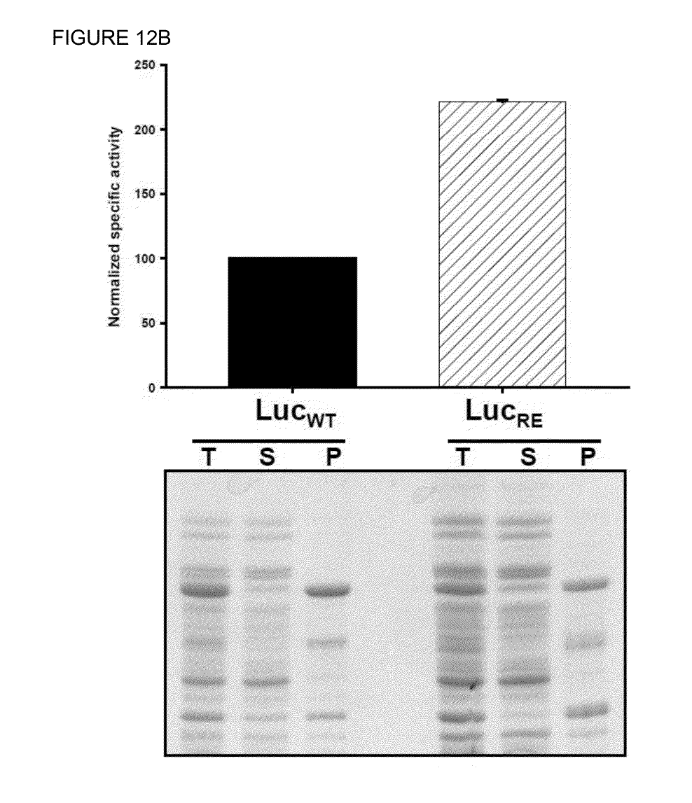

Translation speed harmonization leads to enhanced folding efficiency of identical polypeptides. Our synonymous codon substitutions have allowed us to uncover principles by which sequence composition can affect polypeptide elongation rates and folding. However, in vivo, messages are not uniformly slow or fast (FIG. 8). Rather, the ribosome alternatively accelerates and decelerates as it moves along a given mRNA, which is presumably reflected by the peaks and valleys in our profiles (FIG. 8), corresponding to the presence of clusters of faster and slower codons, respectively. We next asked whether recreating these naturally occurring variations in translation speed by implementing the synonymous sequence-based manipulations described above might enhance the folding efficiency of eukaryotic proteins produced in E. coli. Because, as shown above (FIG. 7) and previously, tRNA gene content differs between bacteria and eukaryotes, a codon with abundant tRNA content in the firefly (predicted to be a “fast” codon by our metric) may correspond to a codon lacking strict Watson-Crick-decoding tRNA genes in E. coli (i.e., predicted to be a “slow” codon in E. coli). We thus employed a harmonization strategy to recode the firefly luciferase sequence in which codons predicted to be translated fast in the fruit fly D. melanogaster (a species evolutionarily close to the firefly whose entire tRNA gene set is known) were matched with synonymous codons predicted to be translated fast in E. coli. Conversely, codons predicted to be translated by non-Watson-Crick tRNAs in the fruit fly were matched by codons with no matching tRNA genes in E. coli. The resulting construct (Luc_{RE}) was utilized to produce recombinant luciferase in E. coli cells. Although the predicted average translation speed of the Luc_{RE} sequence is very similar to that of Luc_{WT}, the luciferase protein produced from the former folded with higher efficiency (FIG. 12). At equivalent amounts of total recombinant protein, luciferase produced from Luc_{RE} consistently displayed increased solubility and about a three-fold higher activity. Thus, it appears that subtle manipulations of ribosome movement along a recombinant mRNA molecule that mimic its movement in the original host appear to be a robust method to improve the folding efficiency of certain eukaryotic proteins that these sequences are indeed translated approximately twice as fast as the non-engineered sequence by performing pulse-chase analyses in living E. coli cells. In agreement with previous results from our laboratory, sequences encoding proteins of eukaryotic origin that were translated at faster rates led to the production of polypeptides that folded with decreased efficiencies. We utilized the knowledge of the available pool of tRNAs in E. coli (based on its tRNA gene content) to redesign the sequence of the model protein firefly luciferase (which folds poorly in bacteria) in an attempt to compel the E. coli ribosome to move at similar segmental rates along the mRNA that the original eukaryotic ribosome would in the original host. This sequence led to a significant increase in the folding efficiency of this model protein, as reflected by its increased solubility and specific activity upon production in E. coli.

How can subtle differences polypeptide elongation rates impact the folding of the polypeptide emerging from the ribosome? Although 2-3 fold differences in the rates of ordinary reactions might not be generally considered significant from a chemical kinetics point of view, a 2-3 fold difference in the rate of synthesis of a protein may have profound biological consequences. For example, a subtle increase in the concentration of a partially folded, aggregation-prone polypeptide intermediate during translation may exceed the critical concentration of the intermediate and lead to its nucleation-dependent aggregation, thus forming intracellular aggregates. In essence, our findings that variations in translation rates impact protein folding support the notion that not all proteins fold globally, but rather follow particular pathways throughout the available structural space, influenced by the speed at which they emerge vectorially from the ribosome.

This analysis finds applications in a variety of fields and settings, including improvements in the production of recalcitrant proteins for vaccine development, recombinant pharmaceuticals and structure-determination studies. Moreover, these results provide further insight into how so-called “silent” polymorphisms can result in human disease₃₉ and on how physiological and disease-related variations in tRNA concentrations impact cellular proteostasis, critical in a wide variety of oncologic, cardiovascular and neurodegenerative disorders.

### Materials and Methods

Prediction of polypeptide elongation rates. In order to assign a translation speed index (i) to each of the 61 codons in a given organism, the following rules were assigned regarding the nature codon(N₁N₂N₃):anticodon(N₃₄N₃₅N₃₆) interactions (where N₁N₂N₃ represents each codon along the 5′→3′ direction in an mRNA and N₃₄N₃₅N₃₆ represents the 5→3′ anticodon loop of the decoding tRNA): (1) Watson-Crick interactions are allowed to occur between N₁N₂G₃:C₃₄N₃₅N₃₆, N₁N₂C₃:G₃₄N₃₅N₃₆, N₁N₂A₃:U₃₄N₃₅N₃₆, N₁N₂U₃:A₃₄N₃₅N₃₆ and N₁N₂C₃:I₃₄N₃₅N₃₆ (where I₃₄ represents inosine, derived from post-transcriptional deamination of some A₃₄-bearing tRNAs); (2) non-Watson-Crick interactions are allowed to occur between N₁N₂G₃:U₃₄N₃₅N₃₆, N₁N₂U₃:G₃₄N₃₅N₃₆, N₁N₂U₃:I₃₄N₃₅N₃₆, and N₁N₂A₃:I₃₄N₃₅N₃₆. Inosination was assumed to occur for all A₃₄-bearing tRNAs in eukaryotes and for A₃₄-bearing tRNAs that decode Arg codons in bacteria. Since a U₃₄A₃₅C₃₆-bearing species of tRNA is generally utilized to decode AUA codons in bacteria, it was assumed that a U₃₄A₃₅C₃₆-bearing tRNAs would partition equally for decoding AUG and AUA codons. In order to obtain normalized values for tRNA gene abundances across organisms for each codon, we divided the number of tRNA genes for every codon by the total number of tRNA genes in the respective synonymous codon group. These values (termed NNN_{%} for each codon) were then utilized, according to the above assumptions, to calculate a translation speed index (i) for each codon (termed NNNᵢ) in a given organism according to the following formulas (where w is a “penalizing” factor for non-Watson-Crick interactions; in this study, w=3 for all such interactions, as these have been experimentally shown to result in ˜3-fold slower polypeptide elongation rates: (1) For all bacterial codons (except those for Ile, Met and Arg): NNUᵢ=NNU_{%}+NNC_{%}/w; NNCᵢ=NNC_{%}; NNAᵢ=NNA_{%}; NNGᵢ=NNA_{%}/w. (2) For bacterial Ile: AUUᵢ=AUU_{%}+AUC_{%}/w; AUCᵢ=AUC_{%} and AUAᵢ=AUG_{%}/w*2. (3) For bacterial Met: AUGᵢ=AUG_{%}/2. (4) For bacterial Arg: treat as a eukaryotic Arg. (5) For eukaryotic two-codon groups and both similar codons of six-codon groups: NNUᵢ=NNU_{%}+NNC_{%}/w; NNCᵢ=NNC_{%}+NNU_{%}; NNAᵢ=NNA_{%}; NNGᵢ=NNG_{%}+NNA_{%}/w. (6) For eukaryotic four-codon groups, the four similar codons of six-codon groups and Ile: NNUᵢ=NNU_{%}/w+NNC_{%}/w; NNCᵢ=NNC_{%}+NNU_{%}; NNAᵢ=NNA_{%}+NNU_{%}/w; NNGᵢ=NNG_{%}+NNA_{%}/w. Once these values were obtained for each organism, they were assigned to the corresponding codons of any protein coding sequence. From the start of the coding sequence, i values of 30 consecutive codons were added and the average value plotted at position number 15. The same operation was performed repeatedly by sliding the window of 30 values one codon position at a time, until of the coding sequence was reached. The resulting i values were plotted as a function of codon position.

Coding sequence engineering. Luciferase mRNAs were engineered as follows: for sequences to be translated slowly (Luc_{slow}), codons that lack isoacceptor tRNA genes in E. coli were selected for each amino acid, with the exception of methionine and tryptophan. If genes for all the anticodons of a particular amino acid are present, then the codon with the least amount of available anticodon interactions at the wobble position was selected. For sequences to be translated faster (Luc_{fast}), codons with the highest number of isoacceptor tRNA genes were selected. In cases were more than one codon had the highest number of tRNA genes, the codon with the highest number of available anticodon interactions at the wobble position was selected. Similarly, Luc_{cbf} was designed to harbor codons for that are the most frequently used in E. coli. Sequences for Luc_{WT-fast} and Luc_{WT-cbf} contain nucleotides 1-50 from Luc_{WT} and the remaining nucleotides from Luc_{fast} and Luc_{cbf}, respectively.

Strains and growth conditions. The E. coli utilized here was BL21 (New England Biolabs). For recombinant protein production (see below), this strain was transformed with the following T7 promoter-based plasmids: pLuc_{WT} (encoding Luc_{WT} with a C-terminal c-myc-His₆ epitope tag), pLuc_{slow}, pLuc_{fast}, pLuc_{cbf}, pLuc_{WT-fast} and pLuc_{WT-cbf}. For activity measurements, cells were grown in LB broth at 37° C. with 250 rpm orbital shaking in volumes that occupied at most one fourth of the total vessel volume, in the presence of ampicillin (100 μg/ml). For the experiments in FIG. 11, volumes of cells containing equivalent A₆₀₀ values were harvested by centrifugation and lysed by spheroplasting₅₀ under native conditions and separated by centrifugation into soluble and pellet fractions₅₁. Similar amounts of total protein in the resulting lysates were verified by the Bradford assay (BioRad). For pulse-chase analysis, cells were grown in a methionine free defined medium (Teknova) at 37° C. with 250 rpm orbital shaking in volumes that occupied at most one fourth of the total vessel volume, in the presence of ampicillin (100 μg/ml).

Recombinant protein production. Starter cultures were grown overnight as described above and diluted the next day. Protein expression was induced at A₆₀₀=0.4 with 1 mM IPTG and harvested at 10 min intervals for activity measurements and at 5 second intervals for pulse-chase analysis. Total amounts of recombinant protein produced during each interval were assessed by examining equivalent amounts of cells (equal A₆₀₀ values), which were subsequently lysed, ran on SDS-PAGE and Coomassie brilliant blue-stained. Aliquots harvested at time points containing equivalent levels of each recombinant protein produced were then lysed under native conditions as described and their solubility and activity assessed as stated below.

Determination of protein solubility. Cells were harvested by centrifugation and spheroplasts were prepared as described₅₀. Spheroplasts were lysed by dilution into an equal volume of native lysis buffer (5 mM MgSO4, 0.2% (v/v) Triton X-100 (Sigma), Complete EDTA-free protease inhibitors (Roche), 100 units/ml Benzonase (Roche), 50 mM Tris-HCl, pH 7.5). Aliquots were centrifuged into supernatant and pellet fractions (20,000 g for 10 min) and analyzed by SDS-PAGE followed by staining with Coomassie brilliant blue as described.

Determination of luciferase activity. Lysates from cells expressing Luc_{WT}, Luc_{fast}, Luc_{cbf} were prepared as above and equivalent dilutions to those used for solubility assessment were utilized. Luciferase activity was determined using the Luciferase Assay System (Promega) in a Sirius luminometer (Berthold) as described.

Pulse-chase analysis. Pulse-chase experiments were performed as described. Cells expressing the desired construct were grown and protein expression was induced as described above. At time 0, (30 minutes post-induction), ³⁵S-Met was added to the culture and 10 seconds later, excess unlabeled Met was added. Aliquots were taken every 5 seconds and placed in ice-cold tubes containing chloramphenicol (200 μg/ml final). Cells were harvested and lysates were run on SDS-PAGE followed by autoradiography.

Calculation of average polypeptide elongation rates. Polypeptide elongation rates were calculated essentially as described. In our constructs, there are 14 methionine residues at positions 92, 187, 189, 320, 336, 389, 421, 433, 467, 495, 518, 526, 555 and 584 from the C′ terminus. The theoretical appearance of radiolabeled methionines was calculated for translation speeds of 5, 10 and 20 aa/s.

1. A method of adjusting the translation kinetics of an mRNA, the method comprising:
selecting a codon from a wobbie codon pair, wherein within the pair, a codon that has G·C or I:C pairing with a cognate anticodon in the third position has increased speed of translation relative to a codon that has G·U or I:U pairing with a cognate anticodon in the third position.

2. The method of claim 1, wherein the wobble codon pair has G·C and G·U pairing.

3. The method of claim 1, wherein a starting polynucleotide sequence is substituted in at least one codon with respect to a wobble codon pair.

4. The method of claim 3, wherein said starting polynucleotide sequence is substituted in at least one member of a wobble codon pair to provide for G·C pairing to increase or decrease translation rate.

5. The method of claim 4, wherein said starting polynucleotide sequence is substituted in at least 20% of the codons that are wobble codons.

6. The method of claim 4, wherein said starting polynucleotide sequence is substituted in at least 50% of the codons that are wobble codons.

7. The method of claim 3, wherein the speed of translation is altered at least 10%.

8. A method of de novo synthesis of a polynucleotide encoding a polypeptide of interest, the method comprising:
selecting a codon for each amino acid of the polypeptide of interest, wherein at least one codon is selected from a wobble codon pair, wherein within the pair, a codon that has G·C or I:C pairing with a cognate anticodon in the third position has increased speed of translation relative to a codon that has G·U I:U pairing with a cognate anticodon in the third position; and
synthesizing the nucleotide sequence.

9. The method of claim 8, characterized in that the step of synthesizing the optimized nucleotide sequence takes place in a device for automatic synthesis of nucleotide sequences which is controlled by the computer that optimizes the nucleotide sequence.

10. A method of producing a kinetic map of a coding sequence of interest, the method comprising:
determining a translation speed index for each of the 61 codons in a given organism where the translation speed index includes a weighting factor w, where w is a “penalizing” factor for non-Watson-Crick interactions;
compiling the translation speed index at each codon, to provide a translation kinetic map.

11. The method of claim 10, wherein w is experimentally determined by calculating ribosome occupancy.

12. The method of claim 10, further comprising:
determining the translation speed index of a second organism (i₂), and
utilizing (i₂) to design a harmonized polynucleotide sequence in which the kinetic map of the coding sequence of interest is harmonized to the second organism.

13. The method of claim 12, further comprising synthesizing the harmonized polynucleotide sequence.

14. The method of claim 13, further comprising operably joining the harmonized polynucleotide sequence to a promoter.

15. A method of producing a polypeptide, the method comprising:
expressing an mRNA according to claim 4, wherein the encoded polypeptide is produced.

16. The method of claim 15, wherein the mRNA is translated in a cell or a cell-free system utilizing a cellular extract.

17. The method of claim 16, wherein the cell or cell-free extract is other than the source organism for the sequence of interest.

18. A polynucleotide produced by the method set forth in claim 1.
